(19) United States
(12) Patent Application Publication
(10) Pub. No.: US 2003/0215840 A1
(43) Pub. Date: Nov. 20, 2003

(54) Methods and compositions for treating cardiovascular disease using 1682, 6169, 6193, 7771, 14395, 29002, 33216, 43726, 69292, 26156, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecules

(75) Inventors: Thomas J. Logan, Springfield, PA (US); Miyoung Chun, Belmont, MA (US); Katherine M. Galvin, Jamaica Plain, MA (US); Aileen Healy, Medford, MA (US); Susan L. Acton, Lexington, MA (US); Mary A. Donoghue, West Roxbury, MA (US); Nancy Stagliano, North Reading, MA (US); Jacqueline Perodin, Arlington, MA (US); Amelie Rodrigue-Way, Malden, MA (US)

Correspondence Address: Jean M. Silveri, MILLENNIUM PHARMACEUTICALS, INC., 75 Sidney Street, Cambridge, MA, 02139, US

(73) Assignee: Millennium Pharmaceuticals, Inc.

(21) Appl. No.: 10/353,690

(22) Filed: Jan. 29, 2003

Related U.S. Application Data
(60) Provisional application No. 60/353,224, filed on Feb. 1, 2002.
(60) Provisional application No. 60/364,529, filed on Mar. 15, 2002.
(60) Provisional application No. 60/373,861, filed on Apr. 19, 2002.
(60) Provisional application No. 60/376,287, filed on Apr. 29, 2002.
(60) Provisional application No. 60/388,080, filed on Jun. 12, 2002.
(60) Provisional application No. 60/390,971, filed on Jun. 24, 2002.
(60) Provisional application No. 60/394,130, filed on Jul. 3, 2002.
(60) Provisional application No. 60/394,797, filed on Jul. 10, 2002.
(60) Provisional application No. 60/404,904, filed on Aug. 21, 2002.
(60) Provisional application No. 60/405,450, filed on Aug. 23, 2002.
(60) Provisional application No. 60/408,070, filed on Sep. 4, 2002.
(60) Provisional application No. 60/424,300, filed on Nov. 6, 2002.
(60) Provisional application No. 60/431,079, filed on Dec. 5, 2002.
(60) Provisional application No. 60/431,042, filed on Dec. 5, 2002.

(51) Int. Cl. A61K 48/00; A61K 31/00; C12Q 1/68; G01N 33/53; G01N 33/567; A61K 39/395

(52) U.S. Cl. 435/6; 435/7.2; 424/146.1; 514/1; 514/44; 514/2

(57) ABSTRACT

The present invention relates to methods for the diagnosis and treatment of cardiovascular disease, including, but not limited to, atherosclerosis, reperfusion injury, hypertension, restenosis, arterial inflammation, heart failure, thrombosis and endothelial cell disorders. Specifically, the present invention identifies the differential expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 and 6585 genes in cardiovascular disease states, relative to their expression in normal, or non-cardiovascular disease states, and/or in response to manipulations relevant to cardiovascular disease. The present invention describes methods for the diagnostic evaluation and prognosis of various cardiovascular diseases, and for the identification of subjects exhibiting a predisposition to such conditions. The invention also provides methods for identifying a compound capable of modulating cardiovascular disease. The present invention also provides methods for the identification and therapeutic use of compounds as treatments of cardiovascular disease.

### RELATED APPLICATIONS

The present application claims the benefit of U.S. Provisional Application serial No. 60/353,224, filed on Feb. 1, 2002, of U.S. Provisional Application serial No. 60/364,529, filed on Mar. 15, 2002, of U.S. Provisional Application serial No. 60/373,861, filed on Apr. 19, 2002, of U.S. Provisional Application serial No. 60/376,287, filed on Apr. 29, 2002, of U.S. Provisional Application serial No. 60/388,080, filed on Jun. 12, 2002, of U.S. Provisional Application serial No. 60/390,971, filed on Jun. 24, 2002, of U.S. Provisional Application serial No. 60/394,130, filed on Jul. 3, 2002, of U.S. Provisional Application serial No. 60/394,797 filed on Jul. 10, 2002, of U.S. Provisional Application serial No. 60/404,904, filed on Aug. 21, 2002, of U.S. Provisional Application serial No. 60/405,450, filed on Aug. 23, 2002, of U.S. Provisional Application serial No. 60/408,070, filed on Sep. 4, 2002, of U.S. Provisional Application serial No. 60/424,300, filed on Nov. 6, 2002, of U.S. Provisional Application serial No. 60/431,079, filed on Dec. 5, 2002, and of U.S. Provisional Application serial No. 60/431,042, filed on Dec. 5, 2002. The entire contents of these provisional patent applications are hereby incorporated by this reference.

### BACKGROUND OF THE INVENTION

Cardiovascular disease is a major health risk throughout the industrialized world. Atherosclerosis, the most prevalent of cardiovascular diseases, is the principal cause of heart attack, stroke, and peripheral vascular disease resulting in significant disability and limb loss, and thereby the principle cause of death in the United States.

Atherosclerosis is a complex disease involving aspects of lipid metabolism and vascular inflammation. Both have significant effects on the initiation and progression of atherosclerosis. Irregular lipid metabolism is a very well established risk factor for atherosclerosis. Elevated low density lipoprotein (LDL), very low density lipoproteins (VLDL), triglycerides and low levels of high density lipoproteins (HDL) all independently contribute to atherosclerosis development and/or progression. There are a number of effective therapies currently being utilized in the clinic that result in lowering of these risk factors and, in turn decrease the rate of mortality and morbidity associated with atherosclerotic disease. Some of these therapies include the cholesterol lowering drugs statins, the triglyceride lowering drugs fibrates and niacin and the triglyceride lowering/HDL raising PPAR alpha activators. There is a need to identify new targets for atherosclerosis therapy.

There have been significant advances made in understanding the role that inflammation plays in the process of atherosclerosis. Atherosclerosis involves many cell types and molecular factors (described in, for example, Ross (1993) Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of many conditions that predispose an individual to accelerated development of atherosclerotic cardiovascular disease. There has been considerable effort in establishing that hypertension contributes to atherosclerosis. The identification of molecules that regulate blood pressure and vascular tone will be useful in discovering new therapies to treat cardiovascular diseases such as atherosclerosis.

### DETAILED DESCRIPTION OF THE INVENTION

The present invention provides methods and compositions for the diagnosis and treatment of cardiovascular disease. As used herein, disorders involving the heart, or “cardiovascular disease” or a “cardiovascular disorder” include a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

A cardiovascular disease can also include thrombosis. Thrombosis can result from platelet dysfunction, e.g. seen in myocardial infarction, angina, hypertension, lipid disorders, diabetes mellitus; myelodysplastic syndromes; myeloproliferative yndromes (including polycythermia vera and thombocythemia); thrombotic thrombocytopenic purpuras; HIV-induced platelet disorders (AIDS-Thrombocytopenia); heparin induced thrombocytopenia; mural cell alterations/interactions leading to platelet aggregation/degranulation, vascular endothelial cell activation/injury, monocyte/macrophage extravasation and smooth muscle cell proliferation; autoimmune disorders such as, but not limited to vasculitis, antiphospholipid syndromes, systemic lupus erythromatosis; inflammatory diseases, such as, but not limited to immune activation; graft Vs host disease; radiation induced hypercoagulation; clotting factor dysregulation either hereditary (autosomal dominant or recessive) such as, but not limited to clotting factor pathways including protein C/S, Anti-thrombin III deficiency, and the Factor V Leiden mutation or acquired such as but not limited to autoimmune, cancer-associated and drug-induced dysregulation of clotting factors.

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes and antisense oligonucleotides. Representative molecules are described herein.

The present invention is based, at least in part, on the discovery that nucleic acid and protein molecules, (described infra), are differentially expressed in cardiovascular disease states relative to their expression in normal, or non-cardiovascular disease states. The modulators of the molecules of the present invention, identified according to the methods of the invention can be used to modulate (e.g., inhibit, treat, or prevent) or diagnose cardiovascular disease, including, but not limited to, atherosclerosis and thrombosis.

“Differential expression”, as used herein, includes both quantitative as well as qualitative differences in the temporal and/or tissue expression pattern of a gene. Thus, a differentially expressed gene may have its expression activated or inactivated in normal versus cardiovascular disease conditions (for example, in an experimental cardiovascular disease system such as in an animal model for atherosclerosis). The degree to which expression differs in normal versus cardiovascular disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic cardiovascular disease, e.g., artheroscierosis and/or thrombosis, evaluation, or may be used in methods for identifying compounds useful for the treatment of cardiovascular disease, e.g., atherosclerosis and/or thrombosis. In addition, a differentially expressed gene involved in cardiovascular disease may represent a target gene such that modulation of the level of target gene expression or of target gene product activity will act to cure, heal, alleviate, relieve, alter, remedy, ameliorate, improve or affect a cardiovascular disease condition, e.g., atherosclerosis and/or thrombosis. Compounds that modulate target gene expression or activity of the target gene product can be used in the treatment of cardiovascular disease. Although the genes described herein may be differentially expressed with respect to cardiovascular disease, and/or their products may interact with gene products important to cardiovascular disease, the genes may also be involved in mechanisms important to additional cardiovascular cell processes.

Molecules of the Present Invention

Gene ID 1682

The human 1682 sequence (SEQ ID NO:1), (GI:340010), known also as human dual specificity protein kinase (TTK or PYT), is approximately 3866 nucleotides long including untranslated regions. The coding sequence, located at about nucleic acid 1026 to 3551 of SEQ ID NO:1, encodes a 841 amino acid protein (SEQ ID NO: 2) (GI:340011).

As assessed by TaqMan analysis, 1682 mRNA expression was detected in megakaryocytic and erythroid lineages in vitro and in bone marrow megakaryocytes (CD41+ cells). Little or no 1682 mRNA expression was observed in the major human organs, i.e., heart, lung, liver, kidney and spleen. Higher levels of 1682 mRNA was observed in the platelets of patients with acute coronary syndromes (myocardial infarct and unstable angina) that also have a history of diabetes as compared with platelets from patients with no history of diabetes and also with normal, age-matched, volunteers.

Signal transduction via serine/threonine and/or tyrosine kinases has been implicated in platelet reactivity, a key component of thrombosis associated with acute coronary syndromes [Circulation. 2000. 102(16):1924-1930]. Evidence exists suggesting that the platelets of diabetic patients are more reactive than normal platelets [Thrombosis Research. 1998. 90(4): 181-90]. 1682 is stimulated by the cytokine tumor necrosis factor (TNF) [Cytokine. 2000. 12(2): 142-150], and elevated serum TNF levels occur in patients with diabetes. Collectively, these data indicate that the increased TNF levels in diabetic patients stimulate increased 1682 expression, thereby contributing to the platelet reactivity and the acute coronary syndrome. Due to these observations and the expression pattern of 1682 in megakaryocytic and erythroid lineages in vitro, modulators of 1682 activity would block platelet reactivity and thus be useful as therapeutics in treating thrombosis and thrombotic conditions. 1682 polypeptides of the present invention are useful to screen for modulators of 1682 activity

Gene ID 6169

The human 6169 sequence (SEQ ID NO:3), (GI:1930074), known also as C-4 methyl sterol oxidase (DESP4), is approximately 1751 nucleotides long including untranslated regions. The coding sequence, located at about nucleic acid 27 to 908 of SEQ ID NO:3, encodes a 293 amino acid protein (SEQ ID NO: 4) (GI:1930075).

As assessed by TaqMan analysis, 6169 mRNA was expressed at high levels in human liver, as compared to other human tissues tested. 6169 mRNA was regulated by cholestyramine in a marmoset model of atherosclerosis. 6169 mRNA was repressed by n-3 polyunsaturated fatty acid in an African Green Monkey model of atherosclerosis.

6169 (C-4 methyl sterol oxidase) is the human ortholog of the yeast gene ERG25 which has been implicated in sterol biosynthesis. Inhibition of C-4 methyl sterol oxidase is predicted to reduce total cholesterol and triglycerides. 6169 mRNA expression was repressed by n-3 polyunsaturated (hypolipidemic diet) and also regulated by cholestyramine and exhibits liver enriched expression. Due to the regulation pattern of 6169 marmoset and African green monkey model atherosclerosis, modulators of 6169 activity would be useful in treating cardiovascular diseases including but not limited to atherosclerosis and hypercholesterolemia. 6169 polypeptides are useful in screening for modulators of 6169 activity.

Gene ID 6193

The human 6193 sequence (SEQ ID NO:5), also known as a GPCR, is approximately 1029 nucleotides long including untranslated regions. The coding sequence, located at about nucleic acid 1 to 1029 of SEQ ID NO:5, encodes a 342 amino acid protein (SEQ ID NO: 6).

As assessed by TaqMan analysis, 6193 mRNA was expressed in human blood vessels and in vessel-rich organs, the highest expression level seen in skeletal muscle. 6193 mRNA expression was also seen in the vasculature in both endothelial and smooth muscle cells. 1

Due to the widespread expression of 6193 in human vasculature, modulators of 6193 activity would modulate vascular tone and thus would be useful in treating cardiovascular disorders, including but not limited to hypertension and those conditions characterized by hypertension. 6193 polypeptides would be useful in screening for modulators of 6193 activity.

Gene ID 7771

The human 7771 sequence (SEQ ID NO:7), (GI:468325), known also as a human phospholipid transfer protein, is approximately 1750 nucleotides long including untranslated regions. The coding sequence, located at about nucleic acid 88 to 1569 of SEQ ID NO:7, encodes a 493 amino acid protein (SEQ ID NO: 8) (GI:468326).

As assessed by TaqMan analysis, 7771 mRNA expression was detected in brain, heart, spleen, placenta, and erythroid and megakaryocytic lineages in vitro. 7771 mRNA was also expressed at highest levels in brain and CD61+ bone marrow megakaryocytes. 7771 mRNA was also detected in platelets from patients with coronary artery disease and at lower levels in platelets from normal volunteers.

7771 protein modulates high density lipoprotein (HDL) particles, converting HDL into larger and smaller particles. Lipoproteins play a critical role in maintaining the phospholipid membrane of platelets and the vessel wall. Lipoproteins are also implicated in maintaining hemostasis and preventing thrombosis. The increased reactivity of platelets from patients with acute coronary diseases results from increased expression of genes such as 7771. Therefore, due to the expression pattern of 7771 in the erythroid and megakaryocytic lineages in vitro and its role in vivo, modulators of 7771 activity would be useful in reducing increased reactivity in the platelets of patients with acute coronary diseases. 7771 polypeptides of the present invention are useful to screen for modulators of 7771 activity.

Gene ID 14395

The human 14395 sequence (SEQ ID NO:9), (GI: 10946200), known also as human neuromedin receptor 1 (NMUR1), is approximately 1212 nucleotides long including untranslated regions. The coding sequence, located at about nucleic acid 1 to 1212 of SEQ ID NO:9, encodes a 403 amino acid protein (SEQ ID NO: 10) (GI:10946201).

As assessed by TaqMan analysis, 14395 mRNA showed restricted exresssion in an organ recital study. The highest expression of 14395 mRNA was observed in breast, adipose, and pancreas. Medium expression of 14395 mRNA was shown in blood vessels, kidney, liver and prostate. Among cardiovascular rich organs (heart, kidney, skeletal muscle and liver), 14395 mRNA was expressed in kidney and liver. There is little or no expression in heart and skeletal muscle. 14395 mRNA was also expressed in all veins and some aorta/arteries. In addition, 14395 mRNA was expressed in laser captured vascular smooth muscle cells in vein.

Due to the widespread expression of 14395 in human vasculature and kidney, modulators of 14395 activity would modulate vascular tone and thus would be useful in treating cardiovascular disorders, including but not limited to hypertension and those conditions characterized by hypertension. 14395 polypeptides would be useful in screening for modulators of 14395 activity.

Gene ID 29002

The human 29002 sequence (SEQ ID NO: 11), known also as a eukaryotic protein kinase, is approximately 2370 nucleotides long including untranslated regions. The coding sequence, located at about nucleic acid 43 to 1338 of SEQ ID NO:11, encodes a 431 amino acid protein (SEQ ID NO: 12).

As assessed by TaqMan analysis, 29002 mRNA was expressed in both in vivo and in vitro samples. 29002 mRNA was expressed in human vasculature. Expression of 29002 mRNA was upregulared in primate atheroma samples compared to normal vessels. In addition, 29002 mRNA was upregulated in macrophages which had been stimulated with CD40 ligand, but not in endothelial cells or smooth muscle cells. In contrast, 29002 mRNA was down-regulated in endothelial cells that were stimulated with mevastatin.

Due to the expression pattern of 29002 in the human vasculature, modulation of 29002 would affect atherognesis and thus be useful as treatment for atherogenesis and atherogenic events. 29002 polypeptides are useful in screening for modulators of 29002 activity.

Gene ID 33216

The human 33216 sequence (SEQ ID NO:13), (GI:4768276), known also as a human long-chain acyl-CoA synthetase or fatty acid transport protein 5 (FATP-5), is approximately 2347 nucleotides long including untranslated regions. The coding sequence, located at about nucleic acid 71 to 2143 of SEQ ID NO:13, encodes a 690 amino acid protein (SEQ ID NO: 14) (GI:4768277).

As assessed by TaqMan analysis 33216 mRNA was expressed in human liver. Expression of 33216 mRNA in the liver was regulated by statin, in vivo model of African Green Monkey, PPAR alpha agonist (a human hepatocyte model), and regulated by a high cholesterol diet in vivo (African Green Monkey model).

33216 (FATP-5) is a member of a family of the Fatty Acid Transport Proteins that has been implicated in lipid metabolism. Inhibition of 33216 is predicted to reduce total cholesterol and triglycerides. Therefore, due to 33216 expression patten in various animal models and its functional role in vivo, modulaotrs of 33216 activity would be useful in treating cardiovascular disorders, including but not limited to atherosclerosis. 33216 polypeptides would be useful in screening for modulators of 33216 activity.

Gene ID 43726

The human 43726 sequence (SEQ ID NO: 15), (GI:3176926), known also as galanin 2 receptor homolog, is approximately 1157 nucleotides long including untranslated regions. The coding sequence, located at about nucleic acid 26 to 1132 of SEQ ID NO:15 encodes a 368 amino acid protein (SEQ ID NO:16) (GI:3176927).

As assessed by TaqMan analysis, 43726 mRNA was expressed in human brain, in megakaryocytic and erythroid lineages in vitro and in bone marrow megakaryocytes (CD61+ cells). 43726 mRNA was also detected in platelets from patients with coronary artery disease but not in platelets from normal volunteers.

Galinin is a neuropeptide that regulates several neural functions including nociception and cognition through ligation with 3 known receptors, galinin receptors 1-3. The mechanism by which the Gal-3 receptor functions is not known, but evidence suggests a role for galinin and galinin receptors in the regulation of neurotrasmitter release [Journal of Biological Chemistry 1998. 273(36):23321-23326]. Due to the expression of 43726 in platelets, and its functional role as the human galanin-3 receptor, it constitutes a parallel pathway may in platelets, implicating 43726 in the control of platelet secretory granule release (degranulation), an important step in thrombus formation. Therefore, modulators of 43726 activity are useful as therapeutics in treating thrombosis and thrombotic conditions. 43726 polypeptides are useful in screening for modulators of 43726 activity.

Gene ID 69292

The human 69292 sequence (SEQ ID NO: 17), (GI: 10334989), known also as potassium-dependent Na/Ca exchanger (NCKX3), is approximately 3763 nucleotides long including untranslated regions. The coding sequence, located at about nucleic acid 38 to 1972 of SEQ ID NO:17, encodes a 644 amino acid protein (SEQ ID NO:18) (GI:14717396).

69292 mRNA was detected by TaqMan analysis in human brain, megakaryocyte precursors (CD34+ cells), in megakaryocytes generated in vitro and in CD41+ bone marrow megakaryocytes. 69292 mRNA was found to be present in platelets, with a relative expression higher in platelets from patients with unstable angina and myocardial infraction as compared with platelets from patients with stable angina and normal volunteers.

Calcium mobilization is a critical component of platelet activation and degranulation. Potassium dependent sodium/calcium exchange activity has been previously demonstrated in platelets [J. Gen. Physiol. 1999. 114:701-711.]. Due to 69292 expression in human brain, megakaryocyte precursors (CD34+ cells), in megakaryocytes generated in vitro and in CD41+ bone marrow megakaryocytes and its funcitional roles as a sodium/calcium exchanger, 69292 is able to regulate intracellular calcium levels in platelets, thereby regulating platelet reactivity. Modulators of 69292 activity would function to regulate platelet activity and would be useful in treating thrombosis and thrombotic conditions. 69292 polypeptides are useful in screening for modulators of 69292 activity.

Gene ID 26156

The human 26156 sequence (SEQ ID NO:19), is approximately 1228 nucleotides long including untranslated regions. The coding sequence, located at about nucleic acid 78 to 728 of SEQ ID NO: 19, encodes a 216 amino acid protein (SEQ ID NO:20).

As assessed by TaqMan analysis, 26156 mRNA was expressed in artery and vein samples. Modulators of 26156 activity are useful in treating cardiovascular diseases. 26156 polypeptides of the present invention are useful in screening for modulators of 26156 activity.

Gene ID 32427

The human 32427 sequence (SEQ ID NO:21), also known as Acyl co A synthase 5 (ACS 5), is approximately 3371 nucleotides long including untranslated regions. The coding sequence, located at about nucleic acid 114 to 2333 of SEQ ID NO:21, encodes a 739 amino acid protein (SEQ ID NO:22).

As assessed by TaqMan analysis, 32427 mRNA was expressed in the human liver. 32427 mRNA was shown to be repressed by cholesterol in monkey liver model. 32427 mRNA was also shown to be up-regulated in human hepatocytes by combination statin/PPAR alpha agonist treatment.

32427 is also known as Acyl co A synthase 5 (ACS 5). Inhibition of 32427 or ACS 5 is predicted to reduce total cholesterol and triglycerides. 32427 is repressed by cholesterol and elevated by the hypolipidemic therapeutic combination of statin/fibrate. This pattern is identical to that observed for genes know to be involved in cholesterol metabolism. Therefore, 32427 has a potential role in cholesterol metabolism/biosynthesis. Due to the 32427 expression in the human liver and various animal models, modulators of 32427 activity are useful in treating cardiovascular diseases. 32427 polypeptides of the present invention are useful in screening for modulators of 32427 activity.

Gene ID 2402

The human 2402 sequence (SEQ ID NO:23), known also as EDG-4 GPCR, is approximately 1734 nucleotides long including untranslated regions. The coding sequence, located at about nucleic acid 85 to 1233 of SEQ ID NO:23, encodes a 382 amino acid protein (SEQ ID NO:24).

As assessed by TaqMan analysis, 2402 mRNA was expressed in the human liver. 2402 mRNA was also repressed by cerivastatin a marmoset model. In a human hepatocyte model, 2402 mRNA was also regulated by statin/PPAR alpha agonist in vitro.

2402 is also known as EDG-4 GPCR. Regulation of 2402 or EDG-4 is predicted to reduce total cholesterol and triglycerides. 2402 expression is repressed by cerivastatin in a marmoset model which predicts a role in cholesterol metabolism/biosynthesis. Due to 2402 mRNA expression in the human liver and marmoset model, modulators of 2402 activity are useful in treating cardiovascular diseases. 2402 polypeptides of the present invention are useful in screening for modulators of 2402 activity.

Gene ID 7747

The human 7747 sequence (SEQ ID NO:25), known also as GMP reductase, is approximately 277 nucleotides long including untranslated regions. The coding sequence, located at about nucleic acid 174 to 260 of SEQ ID NO:25, encodes a 29 amino acid protein (SEQ ID NO: 26).

As assessed by TaqMan analysis, 7747 mRNA was expressed in the human liver. 7747 mRNA was also repressed by cerivastatin a marmoset model.

7747 is known also as GMP reductase. Regulation of 7747 or GMP reductase is predicted to reduce total cholesterol and triglycerides. 7747 expression is repressed by cerivastatin in a marmoset model which predicts a role in cholesterol metabolism/biosynthesis. Due to 7747 mRNA expression in the human liver and marmoset model, modulators of 7747 activity are useful in treating cardiovascular diseases. 7747 polypeptides of the present invention are useful in screening for modulators of 7747 activity.

Gene ID 1720

The human 1720 sequence (SEQ ID NO:27), known also as ZAP70 kinase, is approximately 3151 nucleotides long including untranslated regions. The coding sequence, located at about nucleic acid 286 to 2145 of SEQ ID NO:27, encodes a 619 amino acid protein (SEQ ID NO: 28).

As assessed by TaqMan expression, 1720 mRNA was expressed in the human liver. 1720 mRNA was also shown to be repressed by fenofibrate in vivo and by PPAR alpha selective agonist in vivo.

1720 is also known as ZAP70 kinase. Inhibition of 1720 or ZAP70 kinase is predicted to reduce total cholesterol and triglycerides. 1720 expression is repressed by fenofibrate and a PPAR alpha selective agonist in a marmoset model which predicts a role in triglyceride/cholesterol metabolism. Due to 1720 mRNA expression in the human liver and marmoset model, modulators of 1720 activity are useful in treating cardiovascular diseases. 1720 polypeptides of the present invention are useful in screening for modulators of 1720 activity.

Gene ID 9151

The human 9151 sequence (SEQ ID NO:29), known also as sorbitol dehydrogenase, is approximately 1808 nucleotides long including untranslated regions. The coding sequence, located at about nucleic acid 142 to 1215 of SEQ ID NO:29, encodes a 357 amino acid protein (SEQ ID NO:30).

As assessed by TaqMan analysis, 9151 mRNA was expression in human liver. 9151 mRNA expression was shown to be repressed by statin therapy in vivo, fenofibrate in vivo and PPAR alpha selective agonist in vivo

9151 is also known as sorbitol dehydrogenase. Inhibition of 9151 or sorbitol dehydrogenase is predicted protect against the development of atherosclerosis. 9151 mRNA expression is independently repressed by statin, fenofibrate and a PPAR alpha selective agonist in a marmoset model which predicts a role in triglyceride/cholesterol metabolism. Due to 9151 mRNA expression in the human liver and various animal models, modulators of 9151 activity are useful in treating cardiovascular diseases. 9151 polypeptides of the present invention are useful in screening for modulators of 9151 activity.

Gene ID 60491

The human 60491 sequence (SEQ ID NO:31), known also as a novel glycerol phosphate acytransferase (GPAT), is approximately 2682 nucleotides long including untranslated regions. The coding sequence, located at about nucleic acid 121 to 2448 of SEQ ID NO:31, encodes a 775 amino acid protein (SEQ ID NO: 32).

As assessed by TaqMan analysis, 60491 mRNA was expressed in the human liver. 60491 mRNA expression was also repressed by statin therapy and by fenofibrate in vivo.

60491 is also known as a novel glycerol phosphate acytransferase (GPAT). Inhibition of 60491, which represents a novel Glycerol Phosphate Acyltransferase (GPAT), is predicted to lower triglyceride and/or cholesterol levels. GPAT's are a class of enzymes that have a demonstrated role in triglyceride metabolism. 60491 mRNA expression is independently repressed by statin, and fenofibrate in a marmoset model, which supports a role in triglyceride/cholesterol metabolism for this gene. Due to 60491 mRNA expression in the human liver and various animal models, modulators of 60491 activity are useful in treating cardiovascular diseases. 60491 polypeptides of the present invention are useful in screening for modulators of 60491 activity.

Gene ID 1371

The human 1371 sequence (SEQ ID NO:33), known also as tyrosine kinase BMX, is approximately 2604 nucleotides long including untranslated regions. The coding sequence, located at about nucleic acid 119 to 2212 of SEQ ID NO:33, encodes a 697 amino acid protein (SEQ ID NO: 34).

As assessed by Taqman analysis, 1371 mRNA was expressed in human vessels and endothelial cells. 1371 mRNA was also shown to be expressed in endothelial cells regulated by PPAR alpha agonist (cardioprotective).

1371 is also known as tyrosine kinase BMX. Inhibition of 1371 or BMX kinase is predicted to protect against the development of atherosclerosis. 1371 gene expression is repressed by a PPAR alpha selective agonist in endothelial cells in vitro and 1371 is highly expressed in human arteries (normal and disease), supporting a potential role in the development of atherosclerotic lesions. Due to 1371 mRNA expression in human vessels and endothelial cells, modulators of 1371 activity are useful in treating cardiovascular diseases. 1371 polypeptides of the present invention are useful in screening for modulators of 1371 activity.

Gene ID 7077

The human 7077 sequence (SEQ ID NO:35), known also as a putative kinase, is approximately 6629 nucleotides long including untranslated regions. The coding sequence, located at about nucleic acid 1 to 6416 of SEQ ID NO:35, encodes a 2080 amino acid protein (SEQ ID NO: 36).

As assessed by Taqman analysis, 7077 mRNA was expressed in human vessels and endothelial cells. 7077 mRNA was also shown to be expressed in endothelial cells regulated by PPAR alpha agonist (cardioprotective).

Inhibition of 7077 is predicted to protect against the development of atherosclerosis. 7077 gene expression is repressed by a PPAR alpha selective agonist in endothelial cells in vitro and 7077 is highly expressed in human arteries (normal and disease), supporting a potential role in the development of atherosclerotic lesions. Due to 7077 mRNA expression in human vessels and endothelial cells, modulators of 7077 activity are useful in treating cardiovascular diseases. 7077 polypeptides of the present invention are useful in screening for modulators of 7077 activity.

Gene ID 33207

The human 33207 sequence (SEQ ID NO:37), known also as a putative novel acyltransferase, is approximately 1945 nucleotides long including untranslated regions. The coding sequence, located at about nucleic acid 69 to 1802 of SEQ ID NO:37, encodes a 577 amino acid protein (SEQ ID NO:38).

33207 gene expression was independently repressed by cerivastatin, fenofibrate and a PPAR alpha selective agonist in a marmoset model.

33207 is also known as a putative novel acyltransferase. Inhibition of 33207 is predicted to reduce total cholesterol and triglycerides. 33207 gene expression is independently repressed by cerivastatin, fenofibrate and a PPAR alpha selective agonist in a marmoset model which predicts a role in triglyceride/cholesterol metabolism. Due to 33207 mRNA expression in various animal models, modulators of 33207 activity are useful in treating cardiovascular diseases. 33207 polypeptides of the present invention are useful in screening for modulators of 33207 activity.

Gene ID 1419

The human 1419 sequence (SEQ ID NO:39), known also as ephrin receptor, is approximately 3903 nucleotides long including untranslated regions. The coding sequence, located at about nucleic acid 712 to 3825 of SEQ ID NO:39, encodes a 1037 amino acid protein (SEQ ID NO:40).

As assessed by TaqMan analysis, 1419 mRNA was expressed in smooth muscle, vessel and vein.

1419 is also known as an Ephrin receptor. Inhibition of 1419 is predicted to have beneficial effects in vascular tone/hypertension. 1419 exhibits restricted expression in human smooth muscle cells and human vessel. The expression of 1419 coupled with a potential link to the Rho kinase pathway, known to be involved in vasoactivity, predicts a role for 1419 in vascular tone disease. Due to 1419 mRNA expression in smooth muscle, vessel and vein, modulators of 1419 activity are useful in treating cardiovascular diseases. 1419 polypeptides of the present invention are useful in screening for modulators of 1419 activity.

Gene ID 18036

The human 18036 sequence (SEQ ID NO:41), known also as a calpain 10 protease, is approximately 2180 nucleotides long including untranslated regions. The coding sequence, located at about nucleic acid 183 to 1736 of SEQ ID NO:41, encodes a 517 amino acid protein (SEQ ID NO:42).

As determined by TaqMan analysis, 18036 mRNA expression was shown to be upregulated in the brain, kidney and heart. 18036 mRNA was also found to be upregulated in congestive heart failure (CHF) human tissue samples.

Calpains are cysteine proteases that combine thiol protease activity with calmodulin-like activity. Inhibiting calpain activity leads to attenuated hypoxia-induced cell injury. Increased preload, as seen in pathophysiological states such as heart failure, also induces troponin I degradation independently of myocardial ischemia. Troponin I degradation is a reported marker of myocardial injury in ischemic cornonary syndromes. Due to 18036 mRNA expression in the brain, kindney and heart, along with its functional role, modulators of 18036 activity would be useful in treating disorders associated with cardiovascular disease. 18036 polypeptides of the present invention are useful in screening for modulators of 18036 activity.

Gene ID 16105

The human 16105 sequence (SEQ ID NO:43), known also as a Protein Serine/Threonine Phosphatase family member (PP2C homolog), is approximately 1676 nucleotides long including untranslated regions. The coding sequence, located at about nucleic acid 391 to 1449 of SEQ ID NO:43, encodes a 352 amino acid protein (SEQ ID NO:44).

As determined by TaqMan analysis, 16105 mRNA expression was upregulated in heart and brain. Further TaqMan experiments indicated that 16105 was also upregulated in human congestive heart failure tissue and hypertrophied myocytes compared to the normal tissue. In addition, 16105 mRNA was overexpressed in myocytes which attenutated the hypertrophy phenotype. Yeast two-hybrid experiments demonstrated an interaction of 16105 with hsp27 and/or troponin T.

The hsp27-16105 interaction predicts a role of 16105 in the regulation of actin polymerization which potentially leads to the hypertrophy of myocytes. The troponin T-16105 interaction suggests a role for 16105 in the regulation of the actomyosin ATPase resulting ultimately in a decrease in muscle contraction. Due to 16105 mRNA expression in the brain and heart and its upregulation in congestive heart failure tissue and hypertrophied myocytes, along with its functional role, modulators of 16105 activity would be useful in treating disorders associated with cardiovascular disease, including but not limited to congestive heart failure. 16105 polypeptides of the present invention are useful in screening for modulators of 16105 activity.

Gene ID 38650

The human 38650 sequence (SEQ ID NO:45), known also as an Adenosine Deaminase homolog, is approximately 1680 nucleotides long including untranslated regions. The coding sequence, located at about nucleic acid 340 to 1407 of SEQ ID NO:45, encodes a 355 amino acid protein (SEQ ID NO:46).

As determined by TaqMan analysis, the highest expression of 38650 mRNA was found in heart and skeletal muscles. 38650 mRNA expression was also upregulated in human congestive heart failure (CHF) tissue.

38650 is 22% identical to known Adenosine Deaminase (ADA). ADA catalyses the conversion of adenosine to inosine. Adenosine plasma levels are higher than normal in CHF patients. The elevation of adenosine is potentially cardioprotective and functions to reduce proinflammatory events. Inhibitors of 38650 would potentially function to increase adenosine levels and decrease TNF-alpha levels in the failing heart. In addition, increased adenosine levels in the blood due to the inhibition of 38650 would potentially stimulate other cardioprotective events such as: the inhibition of the growth of cardiac fibroblasts, vasodilation and reduction of inflammatory processes. Due to 38650 mRNA expression in the heart and skeletal muscles, along with its functional role, modulators of 38650 activity would be useful in treating disorders associated with cardiovascular disease. 38650 polypeptides of the present invention are useful in screening for modulators of 38650 activity.

Gene ID 14245

The human 14245 sequence (SEQ ID NO:47), known also as Muscle Specific Serine Kinase (MSSK1), is approximately 1835 nucleotides long including untranslated regions. The coding sequence, located at about nucleic acid 25 to 1626 of SEQ ID NO:47, encodes a 533 amino acid protein (SEQ ID NO:48).

As determined by TaqMan analysis, 14245 mRNA expression was found to be upregulated in human heart and skeletal muscles. 14245 mRNA was also upregulated in human congestive heart failure tissues.

14245 or MSSK1 is homologous to SRPK2 which is a protein kinase found to phosphorylate Ser/Arg rich splicing factors. The potential function of SRPK is in the regulation of pre-mRNA splicing. Several known genes are involved in splicing changes in heart failure tissue samples. These include, but are not limited to, L-type calcium channel alpha Ic, cardiac troponin T, Fas/Apol. Thus, 14245 or MSSK1 potentially plays a role in the splicing changes in genes contributing to congestive heart failure. Due to 14245 mRNA expression in the heart and skeletal muscles, along with its functional role, modulators of 14245 activity would be useful in treating disorders associated with cardiovascular disease. 14245 polypeptides of the present invention are useful in screening for modulators of 14245 activity.

Gene ID 58848

The human 58848 sequence (SEQ ID NO:49), known also as a novel kinase (putative serine/threonine), is approximately 1247 nucleotides long including untranslated regions. The coding sequence, located at about nucleic acid 44 to 1090 of SEQ ID NO:49, encodes a 348 amino acid protein (SEQ ID NO:50).

As determined by TaqMan analysis, 58848 mRNA expression was upregulated in human heart tissue and to a lesser degree in skeletal muscle.

58848 is a Ser/Thr kinase homologous to a zebrafish kinase and to a Ca/calmodulin dependent protein kinase. Due to 58848 mRNA expression in the heart and skeletal muscles, along with its functional role, modulators of 58848 activity would be useful in treating disorders associated with cardiovascular disease. 58848 polypeptides of the present invention are useful in screening for modulators of 58848 activity.

Gene ID 1870

The human 1870 sequence (SEQ ID NO:51), known also as GPR22, is approximately 1881 nucleotides long including untranslated regions. The coding sequence, located at about nucleic acid 237 to 1538 of SEQ ID NO:51, encodes a 433 amino acid protein (SEQ ID NO:52).

As assessed by TaqMan analysis, 1870 mRNA expression was upregulated in human brain and congestive heart failure tissues. Due to 1870 mRNA expression in the brain and heart tissue, modulators of 1870 activity would be useful in treating disorders associated with cardiovascular disease. 1870 polypeptides of the present invention are useful in screening for modulators of 1870 activity.

Gene ID 25856

The human 25856 sequence (SEQ ID NO:53), known also as Pyroglutamyl Peptidase (pyrrolidone-carboxylate peptidase), is approximately 1626 nucleotides long including untranslated regions. The coding sequence, located at about nucleic acid 218 to 808 of SEQ ID NO:53, encodes a 196 amino acid protein (SEQ ID NO:54).

As assessed by TaqMan analysis, 25856 mRNA was expressed in skeletal muscle and was regulated in human congestive heart failure tissue (CHF).

25856 is a pyrrolidone-carboxylate pyroglutamyl peptidase (Pcp) type I. Type I pyroglutamyl peptidases are cytosolic proteins. They are involved in removing 5-oxoproline from various penultimate amino acid residues except L-Proline. Due to 25856 mRNA expression in the heart and skeletal muscles, along with its functional role, modulators of 25856 activity would be useful in treating disorders associated with cardiovascular disease. 25856 polypeptides of the present invention are useful in screening for modulators of 25856 activity.

Gene ID 32394

The human 32394 sequence (SEQ ID NO:55), known also as a voltage-gated potassium channel (KCNQ4), is approximately 2335 nucleotides long including untranslated regions. The coding sequence, located at about nucleic acid 83 to 2170 of SEQ ID NO:55, encodes a 695 amino acid protein (SEQ ID NO:56).

As determined by TaqMan analysis, 32394 mRNA expression was upregulated in the fetal liver, megakaryocytes generated in vitro, the brain and in the heart. 32394 RNA was also expressed in the platelets from patients with coronary artery disease and in particular at higher levels in patients diagnosed with stable angina as compared with patients without coronary artery disease.

Although 32394 or KCNQ4 is found in sensory outer hair cells, and is mutated in dominant deafness [Cell 1999. 96(3):437-46], recent evidence indicates that at least three genes are mutated in deafness. Therefore, affecting the function of 32394 or KCNQ4 as an anti-thrombotic would not have an effect on hearing. The KCNQ4 channels are also implicated in regulating the activity of excitable cells [Am J Physiol Cell Physiol. 2001. 280(4):C859-66]. Since ion fluxes are important regulators of platelet reactivity, the restricted expression in megakaryocytes plus the elevated RNA levels of 32394 in the platelets of patients with stable angina suggests that 32394 plays an important role in the progression from stable to unstable acute vascular lesion. Due to 32394 mRNA expression in the brain, liver and heart, along with its functional role, modulators of 32394 activity would be useful in treating disorders associated with cardiovascular disease, including but not limited to thrombosis and atherosclerosis. 32394 polypeptides of the present invention are useful in screening for modulators of 32394 activity.

Gene ID 3484

The human 3484 sequence (SEQ ID NO:57), known also as a diacylglcerol kinase gamma (DGKγ), is approximately 3758 nucleotides long including untranslated regions. The coding sequence, located at about nucleic acid 517 to 2892 of SEQ ID NO:57, encodes a 791 amino acid protein (SEQ ID NO:58).

As determined by TaqMan analysis, 3484 mRNA expression was upregulated in the brain, megakaryocytes generated in vitro and heart. 3484 mRNA was also expressed at relatively high levels in platelets from patients with and without coronary artery disease and from normal volunteers.

Platelet adhesion to collagen results in platelet activation. Phosphatidic acid levels increase upon platelet adhesion to collagen [Analytical Biochemistry. 1990. 187(1):173-178]. Phosphatidic acid also amplifies the thrombotic response by increasing platelet chemotaxis and aggregation [Platelets. 2001. 12(3):163-170]. 3484 or DGKγ potentially plays a critical role in platelet activation and aggregation following platelet adhesion to collagen via the platelet collagen receptor. Due to 3484 mRNA expression in the brain and heart, along with its functional role, modulators of 3484 activity would be useful in treating disorders associated with cardiovascular disease including but not limited to thrombosis and atherosclerosis. 3484 polypeptides of the present invention are useful in screening for modulators of 3484 activity.

Gene ID 345

The human 345 sequence (SEQ ID NO:59), known also as a melanocortin-1 receptor (MC1-R), a GPCR, is approximately 1270 nucleotides long including untranslated regions. The coding sequence, located at about nucleic acid 169 to 1122 of SEQ ID NO:59, encodes a 317 amino acid protein (SEQ ID NO:60).

As determined by TaqMan analysis, 345 mRNA expression was upregulated the in heart, brain fetal liver, placenta, kidney, in umbilical cord blood, CD34+ cells and megakaryocytes generated in vitro. 345 RNA was also expressed in the platelets from patients with coronary artery disease and in normal volunteers.

The melanocortin-1 receptor or 345 is a Gs-linked receptor. Gs-linked receptors regulate adenyl cyclase, which is an important mediator of platelet activation. The presence of MCI-R in megakaryocytes and platelets indicates that regulating the activity of 345 potentially controls platelet activation and thrombosis. Due to 345 mRNA expression in the heart, brain fetal liver, placenta, kidney, in umbilical cord blood, CD34+ cells and megakaryocytes, along with its functional role, modulators of 345 activity would be useful in treating disorders associated with cardiovascular disease including but not limited to thrombosis and atherosclerosis. 345 polypeptides of the present invention are useful in screening for modulators of 345 activity.

Gene ID 9252

The human 9252 sequence (SEQ ID NO:61), known also as a hydroxymethyltransferase, is approximately 1599 nucleotides long including untranslated regions. The coding sequence, located at about nucleic acid 13 to 1464 of SEQ ID NO:61, encodes a 483 amino acid protein (SEQ ID NO:62).

As determined by TaqMan analysis, 9252 mRNA was upregulated in the human liver when compared to normal liver tissues. Further analysis in a marmoset model showed 9252 regulation by cerivastatin in vivo.

Repression of 9252 reduces total cholesterol and triglycerides. 9252 gene expression regulates the hypolipidemic therapy cerivastatin. This regulation is identical to known genes involved in cholesterol biosynthesis (i.e. HMG CoA reductase). In addition, a known inhibitor of 9252 has lipid lowering effects in an in a rabbit vivo model. Due to 9252 mRNA expression in the liver, along with its functional role, modulators of 9252 activity would be useful in treating disorders associated with cardiovascular disease, including but not limited to atherosclerosis. 9252 polypeptides of the present invention are useful in screening for modulators of 9252 activity.

Gene ID 9135

The human 9135 sequence (SEQ ID NO:63), known also as a fatty aldehyde dehydrogenase, is approximately 1791 nucleotides long including untranslated regions. The coding sequence, located at about nucleic acid 164 to 1621 of SEQ ID NO:63, encodes a 485 amino acid protein (SEQ ID NO:64).

As determined by TaqMan analysis, 9135 mRNA was upregulated in the human liver when compared to normal liver tissues. Further analysis in a marmoset model showed 9135 regulation by cerivastatin in vivo.

Repression of fatty aldehyde dehydrogenase or 9135 potentially reduces total cholesterol and triglycerides. The fatty aldehyde dehydrogenase or 9135 gene is regulated by the hypolipidemic therapy cerivastatin. The regulation of 9135 is identical to known genes involved in cholesterol biosynthesis (i.e. HMG CoA reductase). In addition, fatty aldehyde dehydrogenase or 9135 is implicated in LTB4 degradation. LTB4 is a ligand for PPAR alpha. Inhibition of fatty aldehyde dehydrogenase or 9135 potentially results in increased levels of endogenous PPAR alpha ligand which is beneficial. Due to 9135 mRNA expression in the liver, along with its functional role, modulators of 9135 activity would be useful in treating disorders associated with cardiovascular disease, including but not limited to atherosclerosis. 9135 polypeptides of the present invention are useful in screening for modulators of 9135 activity.

Gene ID 10532

The human 10532 sequence (SEQ ID NO:65), known also as a serine aminotransferase, is approximately 1487 nucleotides long including untranslated regions. The coding sequence, located at about nucleic acid 22 to 1200 of SEQ ID NO:65, encodes a 392 amino acid protein (SEQ ID NO:66).

As determined by TaqMan analysis, 10532 mRNA was upregulated in the human liver when compared to normal liver tissues. Further analysis in a marmoset model showed 10532 regulation by cerivastatin/fenofibrate in vivo. In a human hepatocyte model, 10532 mRNA was also regulated by statin/PPAR alpha agonist in vitro. In addition, 10532 mRNA was also regulated by a high cholesterol diet in vivo.

Repression of serine aminotransferase or 10532 potentially reduces total cholesterol and triglycerides. 10532 gene expression is regulated by the hypolipidemic combination therapy cerivastatin/fenofibrate. 10532 is also regulated by high cholesterol diet. Regulation of 10532 is identical to known genes involved in cholesterol biosynthesis (i.e. HMG CoA reductase). Due to 10532 mRNA liver specific expression, along with its functional role, modulators of 10532 activity would be useful in treating disorders associated with cardiovascular disease, including but not limited to atherosclerosis. 10532 polypeptides of the present invention are useful in screening for modulators of 10532 activity.

Gene ID 18610

The human 18610 sequence (SEQ ID NO:67), known also as melanoma alpha kinase, is approximately 7280 nucleotides long including untranslated regions. The coding sequence, located at about nucleic acid 214 to 5871 of SEQ ID NO:67, encodes a 1885 amino acid protein (SEQ ID NO:68).

As determined by TaqMan analysis, 18610 mRNA was upregulated in heart tissue. 18610, an alpha kinase, functions in the action of calcium mediated cellular responses. The increase in calcium mediated signaling process in vasculature causes vasoconstriction. Therefore, the inhibition of alpha kinases or 18610 potentially results in lowering of calcium mediated signaling, thereby lowering blood pressure. Due to 18610 mRNA expression in the heart, along with its functional role, modulators of 18610 activity would be useful in treating disorders associated with cardiovascular disease, including but not limited to atherosclerosis. 18610 polypeptides of the present invention are useful in screening for modulators of 18610 activity.

Gene ID 8165

The human 8165 sequence (SEQ ID NO:69), known also as an aspartyl aminopeptidase, is approximately 1696 nucleotides long including untranslated regions. The coding sequence, located at about nucleic acid 152 to 1579 of SEQ ID NO:69, encodes a 475 amino acid protein (SEQ ID NO:70).

As determined by TaqMan analysis, the 8165 mRNA was upregulated in the human liver when compared to normal liver tissues. Further analysis in a marmoset model showed 8165 regulation by cerivastatin in vivo. In a human hepatocyte model, 8165 was also regulated by statin/PPAR alpha agonist in vitro.

Repression of aspartyl aminopeptidase potentially reduces total cholesterol and triglycerides. 8165 expression is regulated by the hypolipidemic therapy cerivastatin in vivo (marmosets) and in vitro (human hepatocytes). The regulation of 8165 is identical to known genes involved in cholesterol biosynthesis (i.e. HMG CoA reductase. Due to 8165 mRNA expression in the liver, along with its functional role, modulators of 8165 activity would be useful in treating disorders associated with cardiovascular disease, including but not limited to atherosclerosis. 8165 polypeptides of the present invention are useful in screening for modulators of 8165 activity.

Gene ID 2448

The human 2448 sequence (SEQ ID NO:71), known also as a lysosphingolipid receptor (EDG3), is approximately 2327 nucleotides long including untranslated regions. The coding sequence, located at about nucleic acid 1124 to 2260 of SEQ ID NO:71, encodes a 378 amino acid protein (SEQ ID NO:72).

As determined by TaqMan analysis, 2448 mRNA was highly expressed in human arteries, kidneys and cultured coronary smooth muscle cells. Further TaqMan analysis showed that 2448 mRNA was expressed in cultured human umbilical vein endothelial cells (HUVECs) and in heart tissue. In addition, rat models of vascular tone indicated that 2448 was upregulated in hypertensive rat aortas while downregulated in aortas of rats treated with antihypertensives.

2448 or EDG3, is a lysosphingolipid receptor, with sphingosine 1-phosphate (SIP) as its ligand. SIP and 2448's involment in vascular tone and atherosclerosis is through the signaling cascades that the 2448 receptor triggers. 2448 is Gi, Gq and G12/13 coupled (Ann. Rev. Pharmacol. Toxicol., 2001, 41: 507-534) leading to the activation of Rho, Rac and MAPK signaling pathways while inhibiting the Adenylate cyclase pathway. The resulting intracellular Ca2+ mobilization (J Biochem, 2002, 362; 349-357) and kinase cascade activation plays a role in smooth muscle cell depolarization and the maintenance of vascular tone. Therefore, antagoning 2448 blocks the release of intracellular calcium and promotes hyperpolarization of the cell and vasorelaxation. Due to 2448 mRNA expression in the arteries, kidneys and cultured coronary smooth muscle cells brain, along with its functional role, modulators of 2448 activity would be useful in treating disorders associated with cardiovascular disease, including but not limited to atherosclerosis and hypertension. 2448 polypeptides of the present invention are useful in screening for modulators of 2448 activity.

Gene ID 2445

The human 2445 sequence (SEQ ID NO:73), known also as a lysophosphatidic acid receptor (EDG2), is approximately 1576 nucleotides long including untranslated regions. The coding sequence, located at about nucleic acid 27 to 1121 of SEQ ID NO:73, encodes a 364 amino acid protein (SEQ ID NO:74).

As determined by TaqMan analysis, 2445 mRNA was highly expressed in human arteries, cultured coronary smooth muscle cells and central nervous system (CNS) structures, such as spinal cord (SC), cortex, hypothalamus and dorsal root ganglion (DRG). Further TaqMan analysis indicated that 2445 mRNA was also present at lower levels in heart, kidney, skeletal muscle, adipose and various tissues of the immune system.

2445 or EDG2, is a lysophosphatidic acid receptor, with LPA as its agonist. 2445 and its agonist, LPA, are involved in mutiple intracellular signaling cascades, including those involved in the maintenance of vascular tone and the development of atherosclerosis (Journal of Biological Chemistry, 2000, 2275: 27520-27530). The high expression of 2445 in peripheral blood vessels indicates a role for 2445 in vascular function. 2445 is Gi/o, Gq/11/14 and G12/13 coupled (Ann. Rev. Pharmacol. Toxicol., 2001, 41: 507-534). LPA stimulation leads to activation of the Ras-Raf-ERK pathway, adenylate cyclase inhibition, phospholipase D activation and Ca2+ mobilization. Stimulation of 2445 by LPA mobilizes intracellular Ca2+ via activation of phospholipase C (Molecular Pharmacology, 1998, 54: 881-888). Therefore, antagonizing 2445 blocks the release of intracellular calcium and activation of the ERK and Ras pathways and promotes hyperpolarization of the cell, leading to vasorelaxation. The receptor blockade also protects against smooth muscle cell proliferation which is prevalent in atherosclerosis. Due to 2445 mRNA expression in the arteries, cultured coronary smooth muscle cells, spinal cord, cortex, hypothalamus and dorsal root ganglion, along with its functional role, modulators of 2445 activity would be useful in treating disorders associated with cardiovascular disease including but not limited to atherosclerosis and hypertension. 2445 polypeptides of the present invention are useful in screening for modulators of 2445 activity.

Gene ID 64624

The human 64624 sequence (SEQ ID NO:75), known also as a zinc tranporter (ZIP4), is approximately 2192 nucleotides long including untranslated regions. The coding sequence, located at about nucleic acid 101 to 2044 of SEQ ID NO:75, encodes a 647 amino acid protein (SEQ ID NO:76).

As determined by TaqMan analysis, 64624 mRNA was expressed at the highest levels in kidney and brain. 64624 mRNA was also present at high levels in megakaryocytes generated in vitro. In addition, 64624 mRNA was present in the platelets of patients with coronary artery disease and from normal volunteers.

64624 is a zinc transporter that has been implicated in dietary zinc uptake [AM J Hum Genet. 2002. 71(1):66-73]. Evidence suggests that zinc potentiates the aggregation response via the protein kinase C pathway [J Lab Clin Med. 1994. 123(1):102-109]. Zinc is also an important cofactor for the interaction of platelets with the coagulation mechanism. For example, zinc released from platelets acts as a cofactor for histidine-rich glycoprotein binding to heparin, preventing heparin from interacting with anti-thrombin III and thereby promoting fibrin formation [JBC 1997.272(21): 13541-47]. In addition, zinc is a cofactor of intrinsic coagulation activation. Therefore, regulation of the 64624 would provide a means to inhibit platelet-mediated thrombus formation. Due to 64624 mRNA expression in the kidney and brain, along with its functional role, modulators of 64624 activity would be useful in treating disorders associated with cardiovascular disease, including but not limited to thrombosis and atherosclerosis. 64624 polypeptides of the present invention are useful in screening for modulators of 64624 activity.

Gene ID 84237

The human 84237 sequence (SEQ ID NO:77), known also as a zinc tranporter (hzntl1), is approximately 2952 nucleotides long including untranslated regions. The coding sequence, located at about nucleic acid 202 to 2499 of SEQ ID NO:77, encodes a 765 amino acid protein (SEQ ID NO:78).

As determined by TaqMan analysis, 84237 mRNA was expressed at highest levels in megakaryocytes generated in vitro, in fetal liver and brain. 84237 mRNA was also present at high levels in the platelets of patients with coronary artery disease and from normal volunteers.

84237 or hzntl1 is the human ortholog to a murine zinc transporter, mzntl1. Zinc is an important cofactor for the interaction of platelets with the coagulation mechanism. For example, zinc released from platelets acts as a cofactor for histidine-rich glycoprotein binding to heparin, preventing heparin from interacting with anti-thrombin III and thereby promoting fibrin formation [JBC 1997.272(21):13541-47]. Zinc is also a cofactor of intrinsic coagulation activation. Evidence indicates that zinc potentiates the aggregation response via the protein kinase C pathway [J Lab Clin Med. 1994. 123(1): 102-109]. Therefore, regulation of the 84237, hzntl1, would provide a means to inhibit platelet-mediated thrombus formation. Due to 84237 mRNA expression in megakaryocytes generated in vitro and in fetal liver and brain, along with its functional role, modulators of 84237 activity would be useful in treating disorders associated with cardiovascular disease including but not limited to thrombosis and atherosclerosis. 84237 polypeptides of the present invention are useful in screening for modulators of 84237 activity.

Gene ID 8912

The human 8912 sequence (SEQ ID NO:79), known also as an alkyl-dihydroxyacetonephosphate synthase (alkyl-DHAP synthase), is approximately 2074 nucleotides long including untranslated regions. The coding sequence, located at about nucleic acid 16 to 1992 of SEQ ID NO:79, encodes a 658 amino acid protein (SEQ ID NO:80).

As determined by TaqMan analysis, 8912 mRNA was expressed at the highest levels in brain, in fetal liver and in bone marrow progenitor cells expressing CD15. Further TaqMan experiments indicated that 8912 mRNA was present at high levels in megakaryocytes generated in vitro in kidney and in heart. 8912 mRNA was also present in the platelets from patients with and without coronary artery disease and from normal volunteers.

8912 or alkyl-dihydroxyacetonephosphate synthase (alkyl-DHAP synthase) is a peroxisomal enzyme essential for the biosynthesis of ether phospholipids. Deficiencies in DHAP-synthase affects a specific phospholipid, plasmalogen, reducing its content in the plasma membrane of cells [J.B.C. 1998. 273(17):10296-10301; P.N.A.S. 1997. 94:4475-4480]. Lipid abnormalities exist in patients with diabetes. The platelets of diabetic patients are considered to be more reactive with regard to aggregation and thrombosis. Evidence exists demonstrating that in rodent models of diabetes, plasmalogen levels are altered [Int J. Biochem. 1994. 26(6):759-767]. Altered levels of alkyl-DHAP synthase found in the platelets of diabetic patients with coronary artery disease is found to contribute to the hyper-reactive phenotype associated with these diabetics' platelets. Due to 8912 mRNA expression in brain, in fetal liver and in bone marrow progenitor cells expressing CD15, along with its functional role, modulators of 8912 activity would be useful in treating disorders associated with cardiovascular disease including but not limited to thrombosis and atherosclerosis. 8912 polypeptides of the present invention are useful in screening for modulators of 8912 activity.

Gene ID 2868

The human 2868 sequence (SEQ ID NO:81), known also as a T-cell death associated gene (TDAG8), is approximately 1753 nucleotides long including untranslated regions. The coding sequence, located at about nucleic acid 523 to 1536 of SEQ ID NO:81, encodes a 337 amino acid protein (SEQ ID NO:82).

As determined by TaqMan analysis, 2868 mRNA was predominantly expressed in lymphoid tissues, such as the spleen and tonsil. Further TaqMan experiments showed that 2868 mRNA was also expressed in human arteries and diseased aortas and some central nervous system structures, such as the spinal cord and hypothalamus. In pooled samples of diseased versus normal human arteries, there was a two-fold upregulation of 2868 mRNA in atherosclerotic vessels. In a rat model of hypertension, there was a downregulation of 2868 mRNA in the aortas of animals treated with anti-hypertensives, compared to their vehicle-treated controls.

Stimulation of 2868 results in increases in intracellular calcium and in inhibition of cAMP. Blockade of the 2868 receptor would result in a decreased immune response during the process of atherosclerotic lesion formation and would slow the progression of atherosclerosis. Due to 2868 mRNA expression in the spleen, tonsils, arteries, spinal cord and hypothalamus, along with its functional role, modulators of 2868 activity would be useful in treating disorders associated with cardiovascular disease, including but not limited to atherosclerosis. 2868 polypeptides of the present invention are useful in screening for modulators of 2868 activity.

Gene ID 283

The human 283 sequence (SEQ ID NO:83), known also as a galanin receptor type 1 (Gal1-R), is approximately 1053 nucleotides long including untranslated regions. The coding sequence, located at about nucleic acid 1 to 1050 of SEQ ID NO:83, encodes a 349 amino acid protein (SEQ ID NO:84).

As determined by TaqMan analysis, 283 mRNA was highly expressed in the central nervous system (CNS), particularly the brain and spinal cord. Further Taqman analysis indicated that 283 mRNA was expressed in the human kidneys, arteries and veins. The 283 gene product was also detected in rat tissues and was down-regulated in kidneys of rats which were sensitive to salt-induced hypertension. Our data showed an enrichment of 283 over the other galanin receptors in the kidney.

Galanin stimulation of 283 or Gal 1-R causes a decrease in cAMP in the cell and the opening of inwardly rectifying K+ channels (TiPS, March 2000, 21: 109-117). The high expression of the 283 receptor in CNS structures suggests a central functional role. Our data, describing good expression in peripheral tissues, particularly the kidney and vessels, suggest a novel role for this receptor in cardiovascular function. Therefore, antagonising the 283 receptor would prevent the influx of K+ and increase the intracellular levels in cAMP, promoting hyperpolarization of the cell and vasorelaxation. Due to 283 mRNA expression in the brain, spinal cord, kidney, arteries and veins, along with its functional role, modulators of 283 activity would be useful in treating disorders associated with cardiovascular disease including but not limited to atherosclerosis. 283 polypeptides of the present invention are useful in screening for modulators of 283 activity.

Gene ID 2554

The human 2554 sequence (SEQ ID NO:85), known also as mGlut8 receptor, is approximately 3321 nucleotides long including untranslated regions. The coding sequence, located at about nucleic acid 58 to 2784 of SEQ ID NO:85, encodes a 908 amino acid protein (SEQ ID NO:86).

As determined by TaqMan analysis, 2554 mRNA was expressed in the liver. Further Taqman analysis on specific rodent models of cardiovascular disease indicated that 2554 mRNA was upregulated in vitro in statin/PPAR (peroxisomal proliferators activated receptor) models. 2554 mRNA was also repressed by cholesterol in apolipop protein (apo E) cholesterol models, but upregulated in choletryramine models. 2554 plays a potential role in mediating the effects on hepatic TG. Therefore, inhibiting 2554 potentially has beneficial effects on lipid profiles.

As determined by TaqMan analysis, 2554 mRNA expression was upregulated in 5-fold in failing vs. normal human hearts. The human metabotropic Glutamate receptor type 8 (mGlu8) belongs to the superfamily of G-protein-coupled receptors (GPCRs). This receptor is a member of the group III metabotropic glutamate receptors, which also includes mGlu4, mGlu6 and mGlu7. Glutamate is a neurotransmitter commonly known to produce excitatory effects in the mammalian central nervous system. Scientific literature has also demonstrated that glutamate levels decrease in ischemic hearts. Since mGlu8 can affect intracellular levels of cAMP due to its coupling via Gi, upregulation of the expression of mGlu8 has direct effects on contractility of the heart. Thus, mGlu8 may potentially be implicated in cardiovascular diseases, such as ischemia and heart failure.

Due to 2554 mRNA expression in the liver and in failing human heart, along with its functional role, modulators of 2554 activity would be useful in treating disorders associated with cardiovascular disease, including but not limited to atherosclerosis, dyslipidemia, ischemia and heart failure. 2554 polypeptides of the present invention are useful in screening for modulators of 2554 activity.

Gene ID 9464

The human 9464 sequence (SEQ ID NO:87), known also as an ATP dependent inward rectifying K channel, is approximately 2896 nucleotides long including untranslated regions. The coding sequence, located at about nucleic acid 439 to 1578 of SEQ ID NO:87, encodes a 379 amino acid protein (SEQ ID NO:88).

As determined by TaqMan analysis, 9464 mRNA was expressed in the liver. Further Taqman analysis on specific rodent models of cardiovascular disease indicated that 9464 mRNA was repressed in apolipop protein (apo E) cholesterol models. Published literature indicates that ligands for the 9464 receptor have beneficial lipid lowering effects in pre-clinical models. Therefore, anatagonists and agonists of 9464 potentially have beneficial effects on lipid profiles. Due to 9464 mRNA expression in the liver, along with its functional role, modulators of 9464 activity would be useful in treating disorders associated with cardiovascular disease including but not limited to atherosclerosis and dyslipidemia. 9464 polypeptides of the present invention are useful in screening for modulators of 9464 activity.

Gene ID 17799

The human 17799 sequence (SEQ ID NO:89), known also as a cytidyl transferase, is approximately 1856 nucleotides long including untranslated regions. The coding sequence, located at about nucleic acid 67 to 1236 of SEQ ID NO:89, encodes a 389 amino acid protein (SEQ ID NO:90).

As determined by TaqMan analysis, indicated that 17799 mRNA was expressed in the human liver. Further TaqMan analysis on specific models of cardiovascular disease indicated that 17799 mRNA was repressed in apolipop protein (apo E) cholesterol models and niacin marmoset models. Published literature indicates that the 17799 enzyme is linked to Phosphoethanolamine (PE) biosynthesis and increased PE biosynthesis is associated with increased levels of TG transport from hepatocytes. PE is also pro-thrombotic and PE is a target for glycation, enhancing the atherosclerotic potential of LDL. Inhibition of 17799 potentially has beneficial effects on lipid profiles. Due to 17799 mRNA expression in the human liver, along with its functional role, modulators of 17799 activity would be useful in treating disorders associated with cardiovascular disease including but not limited to atherosclerosis and dyslipidemia. 17799 polypeptides of the present invention are useful in screening for modulators of 17799 activity.

Gene ID 26686

The human 26686 sequence (SEQ ID NO:91), known also as acyl co A synthase, is approximately 3165 nucleotides long including untranslated regions. The coding sequence, located at about nucleic acid 420 to 2582 of SEQ ID NO:91, encodes a 720 amino acid protein (SEQ ID NO:92).

As determined by TaqMan analysis, 26686 mRNA was expressed in the liver. Further Taqman analysis on specific models of cardiovascular disease indicated that 26686 mRNA was upregulated in feno/ceriva marmoset and statin/PPAR (peroxisomal proliferators activated receptor) in vitro hep models, but 26686 mRNA showed a decreased expression leves in monkey cholesterol and apolipop protein (apo E) cholesterol models. Published literature indicates that 26686 plays a role of in lipid metabolism. Therefore, inhibiting 26686 potentially leads to lower lipid levels. Due to 26686 mRNA expression in the liver, along with its functional role, modulators of 26686 activity would be useful in treating disorders associated with cardiovascular disease, including but not limited to atherosclerosis and dyslipidemia. 26686 polypeptides of the present invention are useful in screening for modulators of 26686 activity.

Gene ID 43848

The human 43848 sequence (SEQ ID NO:93), known also as N-acyltransferase, is approximately 1124 nucleotides long including untranslated regions. The coding sequence, located at about nucleic acid 135 to 1025 of SEQ ID NO:93, encodes a 296 amino acid protein (SEQ ID NO:94).

As determined by TaqMan analysis, 43848 mRNA was increased in cholestyramine and fenofibrate/cerivastatin marmoset models, but 43848 expression levels were decreased in apolipop protein (apo E) cholesterol model. Published literature indicates that 43848 is part of the FXR nuclear receptor negative feedback loop for bile acid synthesis. Antagonist for FXR results in cholesterol reduction. Therefore, inhibiting 43848 has beneficial effects on lipid profiles. Due to 43848 mRNA expression in cholestyramine and fenofibrate/cerivastatin marmoset models, along with its functional role, modulators of 43848 activity would be useful in treating disorders associated with cardiovascular disease, including but not limited to atherosclerosis and dyslipidemia. 43848 polypeptides of the present invention are useful in screening for modulators of 43848 activity.

Gene ID 32135

The human 32135 sequence (SEQ ID NO:95), known also as tetrahydrofolate dehydrogenase, is approximately 3040 nucleotides long including untranslated regions. The coding sequence, located at about nucleic acid 111 to 2819 of SEQ ID NO:95, encodes a 902 amino acid protein (SEQ ID NO:96).

As determined by TaqMan analysis, 32135 mRNA was expressed in the human liver. Taqman analysis on specific models of cardiovascular disease indicated that 32135 mRNA was repressed in apolipop protein (apo E) cholesterol, monkey cholesterol and niacin models. Published literature indicates that mutations in methylene tetrahydrofolate reductase associated with elevated homocysteine levels. 32135 plays a potential role in homocysteine biosynthesis. Therefore, inhibiting 32135 has beneficial effects on lipid profiles. Due to 32135 mRNA expression in the liver, along with its functional role, modulators of 32135 activity would be useful in treating disorders associated with cardiovascular disease, including but not limited to atherosclerosis and dyslipidemia. 32135 polypeptides of the present invention are useful in screening for modulators of 32135 activity.

Gene ID 12208

The human 12208 sequence (SEQ ID NO:97), known also as a human small conductance calcium-activated potassium channel protein 3 (KCNN3), is approximately 3095 nucleotides long including untranslated regions. The coding sequence, located at about nucleic acid 334 to 2544 of SEQ ID NO:97, encodes a 736 amino acid protein (SEQ ID NO:98).

As determined by TaqMan analysis, 12208 mRNA was expressed in the spleen, kidney, heart and brain. Further TaqMan analysis indicated that 12208 was expressed at high levels in megakaryocytes generated in vitro. In addition, 12208 RNA is detected at high levels in the platelets of patients with coronary artery disease and in platelets from normal volunteers.

The calcium-activated potassium channel, KCNN3 or 12208 is activated by intracellular calcium. Calcium spikes are an essential component of platelet activation. Increased levels of KCNN3 or 12208 in the platelets of patients with stable angina, indicates an increased platelet reactivity in these patients. Therefore, inhibition of KCNN3 or 12208, provides a means to inhibit platelet aggregation and thrombus formation. Due to 12208 mRNA expression in the spleen, kidney, heart and brain, along with its functional role, modulators of 12208 activity would be useful in treating disorders associated with cardiovascular disease, including but not limited to thrombosis and atherosclerosis. 12208 polypeptides of the present invention are useful in screening for modulators of 12208 activity.

Gene ID 2914

The human 2914 sequence (SEQ ID NO:99), known also as a mosaic serine protease, is approximately 2393 nucleotides long including untranslated regions. The coding sequence, located at about nucleic acid 88 to 1833 of SEQ ID NO:99, encodes a 581 amino acid protein (SEQ ID NO:100).

As determined by TaqMan analysis, 2914 mRNA was expressed at the highest levels in megakaryocytes generated in vitro and in placenta. Further Taqman analysis indicated that 2914 RNA is detected in the platelets of patients with coronary artery disease

The mosaic serine protease or 2914 is spliced with and without a transmembrane domain [BBA 2001. 19;1518(1-2):204-409]. Serine proteases play an essential role in maintaining hemostasis and in promoting thrombogenesis. Therefore, inhibition of the mosaic serine protease, 2914, would provide a means to inhibit thrombus formation. Due to 2914 mRNA expression in megakaryocytes generated in vitro and in the placenta, along with its functional role, modulators of 2914 activity would be useful in treating disorders associated with cardiovascular disease including but not limited to thrombosis and atherosclerosis. 2914 polypeptides of the present invention are useful in screening for modulators of 2914 activity.

Gene ID 51130

The human 51130 sequence (SEQ ID NO:101), known also as an peptidylarginine deiminase (PAD), is approximately 2263 nucleotides long including untranslated regions. The coding sequence, located at about nucleic acid 27 to 2018 of SEQ ID NO: 101, encodes a 663 amino acid protein (SEQ ID NO: 102).

As determined by TaqMan analysis, 51130 mRNA was expressed in hematopoietic cells and mononuclear cells and CD14 positive cells (monocytes). Further TaqMan analysis indicated that 51130 mRNA was present at high levels in megakaryocytes generated in vitro and in the platelets of patients with coronary artery disease and from normal volunteers.

PAD (51130) is a peptidylarginine deiminase that is implicated in myeloid differentiation [JBC. 1999. 274(39):27786-27792]. Recent results indicate that PAD converts an essential arginine residue on antithrombin III (ATIII) to citrulline, thereby negatively affecting the function of this important anticoagulant. Deimination of antithrombin III results in an increased affinity of ATIII for heparin thus inactivating the thrombin-neutralizing function of ATIII [JBC 1997. 272(32): 19652-19655]. The inactivation of ATIII by platelet released PAD or 51130 would create an imbalance in hemostasis by accelerating thrombin activation of platelets and fibrin formation. Therefore, inhibition of the platelet peptidylarginine deiminase provides a means to inhibit platelet-mediated thrombus formation. Due to 51130 mRNA expression in hematopoietic cells and mononuclear cells, along with its functional role, modulators of 51130 activity would be useful in treating disorders associated with cardiovascular disease including but not limited to thrombosis and atherosclerosis. 51130 polypeptides of the present invention are useful in screening for modulators of 51130 activity.

Gene ID 19489

The human 19489 sequence (SEQ ID NO: 103), known also as a novel secreted phospholipase A2 (sPLA2), is approximately 1204 nucleotides long including untranslated regions. The coding sequence, located at about nucleic acid 143 to 712 of SEQ ID NO: 103, encodes a 189 amino acid protein (SEQ ID NO:104).

As determined by TaqMan analysis, 19489 mRNA was expressed in megakaryocytes generated in vitro, in erythroid cells and in heart. In addition, 19489 RNA was present at very high levels in the platelets of patients with coronary artery disease and from normal volunteers

sPLA2 or 19489 is a novel secreted phospholipase A2 with as of yet an undefined function [JBC. 2000. 275(51):39823-39826]. Lysophosphatidic acid is a lipid mediator and platelet agonist. Lysophosphatidic acid also binds and activates endothelial cells. Lysophosphatidic acid is generated by the enzymatic activity of phospholipases. The high levels of sPLA2 or 19489 in platelets and its restricted expression indicate that sPLA2 or 19489 is an important enzyme during acute coronary syndrome. SPLA2 or 19489 is potentially responsible for the generation of lysophosphatidic acid and its increased plasma levels following acute coronary events. Therefore, inhibition of the platelet sPLA2 or 19489, would inhibit platelet-endothelial cell interactions and thrombus formation. Due to 19489 mRNA expression in megakaryocytes generated in vitro, in erythroid cells and in heart, along with its functional role, modulators of 19489 activity would be useful in treating disorders associated with cardiovascular disease including but not limited to thrombosis and atherosclerosis. 19489 polypeptides of the present invention are useful in screening for modulators of 19489 activity.

Gene ID 21833

The human 21833 sequence (SEQ ID NO: 105), known also as the enzyme kynureninase, is approximately 1637 nucleotides long including untranslated regions. The coding sequence, located at about nucleic acid 107 to 1504 of SEQ ID NO:105, encodes a 465 amino acid protein (SEQ ID NO:106).

As determined by TaqMan analysis, 21833 mRNA was expressed at highest levels in the human liver, in macrophages and tonsil. Further TaqMan analysis indicated that 21833 mRNA was upregulated in diseased human arteries when compared to normal vessels. In cultured human monocytes and macrophages, 21833 was upregulated after interferon gamma and CD40-ligand stimulation. By laser capture microdissection and TM analysis, 21833 was enriched in the macrophage and monocyte-rich lesion area of atherosclerotic arteries.

21833 or kynureninase is a downstream enzyme involved in tryptophan metabolism (Saito et al., AJP-Renal Physiology, 279: 3, F565-F572, 2000). Kynureninase converts kynurenine to anthranilic acid and 3-hydroxykynurenine to 3-hydroxyanthranilic acid. The downstream product of 3-hydroxyanthranilic acid metabolism is quinolinic acid formation. Quinolinic acid is an agonist of the NMDA-receptor and is excitotoxic to neural cells; it has been found in macrophages stimulated by interferon gamma and TNFalpha (Chiarugi, A et al., Journal of Neuroimmunology, 120: 1-2, 190-198.) Therefore, inhibiting 21833 or kynureninase leads to the reduction of the toxin, quinolinic acid, which can reduce atherosclerotic injury in vessels. Due to 21833 mRNA expression in the human liver, in macrophages and tonsil, along with its functional role, modulators of 21833 activity would be useful in treating disorders associated with cardiovascular disease, including but not limited to thrombosis and atherosclerosis. 21833 polypeptides of the present invention are useful in screening for modulators of 21833 activity.

Gene ID 2917

The human 2917 sequence (SEQ ID NO:107), known also as serine protease 23, is approximately 1647 nucleotides long including untranslated regions. The coding sequence, located at about nucleic acid 105 to 1256 of SEQ ID NO: 107, encodes a 383 amino acid protein (SEQ ID NO:108).

As determined by TaqMan analysis, 2917 mRNA was expressed at highest levels in human arteries, veins, HUVECs, smooth muscle cells and vascular rich organs. By transcriptional profiling, there was upregulation of 2917 mRNA in response to anti-hypertensive therapy in normotensive rat aortas. By angiotensinl receptor blockade, L-type calcium channel blockade and by ATP-dependent potassium channel opening indicated that 2917 mRNA was upregulated.

Serine protease 23 or 2917 is a novel serine protease cloned from human umbilical vein endothelial cells. Its abundance in vascularized tissues such as the aorta, vein, heart and kidney; its presence in endothelial cells and smooth muscle cells; and its regulation in a rat model of vascular tone gene discovery, implicate 2917 in the maintenance of systemic blood pressure. Therefore, antagonists of 2917 would function in the reduction of peripheral vascular resistance and decrease blood pressure. Due to 2917 mRNA expression in the arteries, veins, HUVECs, smooth muscle cells and vascular rich organs, along with its functional role, modulators of 2917 activity would be useful in treating disorders associated with cardiovascular disease, including but not limited to thrombosis and atherosclerosis. 2917 polypeptides of the present invention are useful in screening for modulators of 2917 activity.

Gene ID 59590

The human 59590 sequence (SEQ ID NO: 109), known also as heart alpha-kinase (HAK), is approximately 5375 nucleotides long including untranslated regions. The coding sequence, located at about nucleic acid 199 to 4794 of SEQ ID NO: 109, encodes a 1531 amino acid protein (SEQ ID NO:110).

As determined by TaqMan analysis, 59590 mRNA was upregulated in the experiments comparing human heart left ventricle samples of 12 congestive heart failure patients (CHF) with 7 non-failing patients (NF, control). 59590 mRNA expression was restricted to heart and skeletal muscle and was expressed to a lesser degree in kidney, osteoblasts and smooth muscle cells.

Heart alpha-kinase (HAK) or 59590 belongs to a new family of kinases (alpha-kinases) that is unlike the conventional serine/threonine/tyrosine kinases. Family members include eukaryotic elongation factor-2 kinase (eEF-2K) and Dictyostelium's myosin heavy chain kinase A, B and C (MHCK A, B and C). HAK or 59590 is highly expressed in heart and skeletal muscle and is regulated in heart failure. Because HAK or 59590 belongs to the same class as Dictyostelium's MHCK A, HAK or 59590 potentially plays a role in sarcomere assembly and in contraction efficiency which is known to be impaired in heart failure patients. Muscle contracts when cytosolic calcium is increased (released from sarcoplasmic reticulum at every cycle) through actomyosin interactions that forms part of the sarcomere. During the progression of heart failure, the myocytes try to compensate for an increase workload by increasing in size (hypertrophy). At the late stage of heart failure, there is loss of contractile elements, marked disruption of Z bands and a severe disruption of the normal parallel arrangement of sarcomere resulting in impaired contraction. The upregulation of HAK or 59590 in heart failure potentially causes the impaired contraction. Therefore inhibitors to HAK or 59590 are beneficial in the treatment of heart failure and other cardiovascular disorders. Due to 59590 mRNA expression in the heart and skeletal muscle, along with its functional role, modulators of 59590 activity would be useful in treating disorders associated with cardiovascular disease, including but not limited to ischemia and heart failure. 59590 polypeptides of the present invention are useful in screening for modulators of 59590 activity.

Gene ID 15992

The human 15992 sequence (SEQ ID NO: 111), known also as cardiolipin synthase, is approximately 1241 nucleotides long including untranslated regions. The coding sequence, located at about nucleic acid 94 to 999 of SEQ ID NO:111, encodes a 301 amino acid protein (SEQ ID NO:112).

As determined by TaqMan analysis, 15992 mRNA showed a 5 fold upregulation in ischemic versus normal heart samples in ischemic heart samples. Further Taqman analysis indicated that 15992 mRNA expresssion was restricted with the highest level in CHF heart followed by lower levels of expression in colon tumor, prostate tumor and normal adrenal gland.

Cardiolipin synthase is a member of this family of transferases and cardiolipin, associated primarily with the inner mitochondrial membrane of mammalian cells, comprises approximately 15% of the entire cardiac glycerolphospholipid mass. Cardiolipin is required for a number of mitochondrial enzymes involved in energy metabolism; content of cardiolipin is important for activation of these enzymes. Ischemic events will decrease the cardiolipin content as well as the level of phosphorylation through cytochrome oxidase. Cardiolipin is enriched in oxidatively sensitive acyl residues, addition of cardiolipin but not peroxidized cardiolipin will almost completely restore activity of cytochrome oxidase. Therefore, the upregulation of 15992 potentially results in either synthesis of an alternate phospholipid that replaces cardiolipin in the mitochondrial membrane or alters its molecular composition resulting in a decrease in cytochrome oxidase activity. Due to 15992 mRNA expression in the brain and heart, along with its functional role, modulators of 15992 activity would be useful in treating disorders associated with cardiovascular disease including but not limited to ischemia and heart failure. 15992 polypeptides of the present invention are useful in screening for modulators of 15992 activity.

Gene ID 2094

The human 2094 sequence (SEQ ID NO: 113), known also as human germinal center kinase (GCK), which is approximately 2906 nucleotides long including untranslated regions. The coding sequence, located at about nucleic acid 40 to 2499 of SEQ ID NO: 113, encodes a 765 amino acid protein (SEQ ID NO: 114).

As determined by TaqMan analysis, 2094 mRNA was expressed at highest levels in brain and in megakaryocytes generated in vitro. 2094 mRNA was also present in the placenta. Further TaqMan analysis indicated that 2094 mRNA was detected in the platelets of apheresed normal donors and in the platelets of patients with coronary artery disease and in platelets from normal volunteers. 2094 mRNA was also present at statistically significant elevated levels in the platelets of patients with angina as compared with patients with no coronary artery disease or normal volunteers.

The germinal center kinase, GCK or 2094, activates at least two MAP kinases, MEKK1 and MLK3 [MCB. 2002. 22(3):737-749]. MLK3 activates MKK4 through a phosphorylation event, and MKK4 activates JNK kinase also by phosphorylation [JBC.2000. 275(36);27893-27900]. JNK activation is required for platelet aggregation. GCK or 2094 also activates MEKK1 inducing autophosphorylation of MEKK1 however, evidence exists indicating that MEKK1 is not a mediator of platelet aggregation [JBC. Sep. 2, 2002 3 9epub ahead of print]. The high levels of 2094 mRNA found in platelets and the elevated levels found in the platelets of patients with angina indicates that 2094 regulates JNK-mediated platelet aggregation. Therefore, inhibiting 2094 provides a means to inhibit platelet-rich thrombus formation. Due to 2094 mRNA expression in the brain and in megakaryocytes, along with its functional role, modulators of activity would be useful in treating disorders associated with cardiovascular disease, including but not limited to thrombosis and atherosclerosis. 2094 polypeptides of the present invention are useful in screening for modulators of 2094 activity.

Gene ID 2252

The human 2252 sequence (SEQ ID NO: 115), known also as a human germinal center kinase III (MASK), is approximately 3335 nucleotides long including untranslated regions. The coding sequence, located at about nucleic acid 221 to 1471 of SEQ ID NO: 115, encodes a 416 amino acid protein (SEQ ID NO: 116).

As determined by TaqMan analysis, 2252 mRNA was expressed in placenta and peripheral blood mononuclear cells. 2252 mRNA was expressed at high levels in megakaryocytes generated in vitro. In addition, 2252 mRNA is detected in the platelets of apheresed normal donors and at high levels in the platelets of patients with coronary artery disease and in platelets from normal volunteers.

2252 is also known as germinal center kinase III subfamily member known as MASK. MASK or 2252 activity has been implicated in apoptosis [JBC. 2002. 277(8):5929-5939]. Recent reports compare the mechanisms driving platelet activation and degranulation to the apoptotic pathway [Blood.1999. 93(12):4222-4231]. Based on the literature and expression data, 2252 or MASK is a required signaling component regulating platelet activation and degranulation. Therefore, inhibition of 2252 or MASK, would inhibit platelet aggregation and thrombus formation. Due to 2252 mRNA expression in placenta, peripheral blood mononuclear cells and megakaryocytes generated in vitro, along with its functional role, modulators of 2252 activity would be useful in treating disorders associated with cardiovascular disease including but not limited to thrombosis and atherosclerosis. 2252 polypeptides of the present invention are useful in screening for modulators of 2252 activity.

Gene ID 3474

The human 3474 sequence (SEQ ID NO: 117), known also as vesicular neurotransmitter transporter (VMAT2), is approximately 1800 nucleotides long including untranslated regions. The coding sequence, located at about nucleic acid 114 to 1658 of SEQ ID NO: 117, encodes a 514 amino acid protein (SEQ ID NO: 118).

As determined by TaqMan analysis, 3474 mRNA was predominantly expressed in megakaryocytes, CD34+ progenitor cells and platelets. Further Taqman analysis also detected 3474 mRNA in normal human ovary.

The vesicular neurotransmitter transporter, VMAT2 or 3474, is known to sequester monoamines within synaptic vesicles. VMAT2 or 3474's expression in platelets indicate a similar function of sequestering monoamines within platelet granules. Platelet granule content is released upon platelet aggregation and is essential to the development of a thrombus. Our data indicates that this function is regulated in the platelets of patients with acute coronary syndromes, myocardial infarction and unstable angina as compared with patients with stable angina. Therefore, regulating 3474 or VMAT2 protects against the development of acute coronary syndromes. Due to 3474 mRNA expression in megakaryocytes, CD34+ progenitor cells and platelets, along with its functional role, modulators of 3474 activity would be useful in treating disorders associated with cardiovascular disease including but not limited to thrombosis and atherosclerosis. 3474 polypeptides of the present invention are useful in screening for modulators of 3474 activity.

Gene ID 9792

The human 9792 sequence (SEQ ID NO: 119), known also as human high-affinity cationic amino acid transporter-1 (CAT-1), is approximately 2157 nucleotides long including untranslated regions. The coding sequence, located at about nucleic acid 148 to 2037 of SEQ ID NO: 119, encodes a 629 amino acid protein (SEQ ID NO: 120).

As determined by TaqMan analysis, 9792 mRNA was expressed at highest levels in red blood cells and in megakaryocytes generated in vitro. Further TaqMan analysis indicated that 9792 mRNA was present in placenta and brain. In addition, 9792 mRNA was present in the platelets of patients with angina and in platelets from normal volunteers.

9792 or CAT-1 is a cationic amino acid transporter involved in the transport of arginine, a precursor to the vasoamine, nitric oxide. Ischemia-reperfusion injury is a serious complication resulting from current treatments for clot resolution in the acute coronary syndromes. While endogenous production of nitric oxide is important for vascular tone, recent evidence suggests controlling the concentration of nitric oxide is essential to preventing vascular injury and arryhthmias [J AM Coll Crdiol. 2001.38(2):546-554]. Regulation of CAT-1 on platelets provides a means to control nitric oxide production and the associated free radical injury following ischemia-reperfusion that occurs during thrombus formation and resolution. Therefore, inhibition of CAT-1 or 9792 protects against secondary coronary events. Due to 9792 mRNA expression in red blood cells and megakaryocytes generated in vitro, along with its functional role, modulators of 9792 activity would be useful in treating disorders associated with cardiovascular disease including but not limited to thrombosis and atherosclerosis. 9792 polypeptides of the present invention are useful in screening for modulators of 9792 activity.

Gene ID 15400

The human 15400 sequence (SEQ ID NO:121), known also as a human germinal center kinase called Traf2 and NCK interacting kinase (TNIK), is approximately 3807 nucleotides long including untranslated regions. The coding sequence, located at about nucleic acid 1 to 3807 of SEQ ID NO: 121, encodes a 1268 amino acid protein (SEQ ID NO: 122).

As determined by TaqMan analysis, 15400 mRNA was expressed in brain and megakaryocytes generated in vitro. 15400 mRNA was detected in the platelets of apheresed normal donors and at high levels in the platelets of patients with coronary artery disease and in platelets from normal volunteers.

The germinal center kinase, 15400 or TNIK, activates the JNK signaling pathway [JBC. 1999. 273(43):30729-30737]. In vitro derived evidence suggests that exposure of platelets to VWF or thrombin activates JNK [Br J Haematol. 2000.109(4):851-856]. 15400 or TNIK is also implicated in actin based cytoskeletal reorganization. 15400 or TNIK phosphorylates gelsolin, the F-actin severing protein, in TNIK transfected cells [JBC. 1999. 273(43):30729-30737]. Shape change is an early step in platelet activation and requires gelsolin activity for actin reorganization [J Cell Biol. 1996.134(2):389-399]. Based on the literature and expression data, 15400 or TNIK plays an early step in platelet activation. Therefore, inhibition of TNIK, 15400, provides a means to inhibit platelet activation and thrombus formation. Due to 15400 mRNA expression in brain and megakaryocytes generated in vitro, along with its functional role, modulators of 15400 activity would be useful in treating disorders associated with cardiovascular disease including but not limited to thrombosis and atherosclerosis. 15400 polypeptides of the present invention are useful in screening for modulators of 15400 activity.

Gene ID 1452

The human 1452 sequence (SEQ ID NO:123), known also as fgr kinase, is approximately 2354 nucleotides long including untranslated regions. The coding sequence, located at about nucleic acid 20 to 2218 of SEQ ID NO: 123, encodes a 529 amino acid protein (SEQ ID NO: 124).

As determined by TaqMan analysis, 1452 mRNA was expressed in a variety of human tissues, including heart, spleen, pancreas, lung and tonsil. Further TaqMan analysis indicated that 1452 mRNA was expressed in macrophages, neutrophils, and erythroid progenitor cells. 1452 mRNA was also expressed in normal and atherosclerotic human arteries. In ApoE knockout mouse aortas, there was a substantial increase in expression of 1452 mRNA expression in the aortic arches compared to abdominal aortas; this expression was highly correlated with CD68 levels, indicating that 1452 mRNA was enriched in the macrophage rich lesion compartment of the vessel. In addition, 1452 mRNA was robustly expressed in cultured human monocytes and macrophages.

1452 is a member of the src family of tyrosine kinases and is also called fgr kinase (Notario et al., J Cell Biol, 1989, 109: 3129-3136). It is thought that 1452 activity is partly responsible for macrophage migration and spreading. Given that macrophage recruitment and infiltration are a major aspect of the disease process in vessel wall atherosclerosis, inhibiting 1452 would result in a reduction of macrophage and eventually foam cell content in an atherosclerotic plaque and a diminished lesion size. Due to 1452 mRNA expression in the heart, spleen, pancreas, lung, tonsil, macrophages, neutrophils, and erythroid progenitor cells, along with its functional role, modulators of 1452 activity would be useful in treating disorders associated with cardiovascular disease, including but not limited to atherosclerosis. 1452 polypeptides of the present invention are useful in screening for modulators of 1452 activity.

Gene ID 6585

The human 6585 sequence (SEQ ID NO:125), known also as acyl peptide hydrolase, is approximately 2374 nucleotides long including untranslated regions. The coding sequence, located at about nucleic acid 20 to 2218 of SEQ ID NO:125 encodes a 732 amino acid protein (SEQ ID NO:126).

As determined by TaqMan analysis, 6585 mRNA was highly expressed in human arteries, umbilical vein endothelial cells, coronary smooth muscle cells, congestive heart failure samples, kidneys, skeletal muscle and brain cortex. There was significant (t-test, p=0.01) upregulation demonstrated across human heart failure samples compared to non-failing ventricles. In rats, 6585 mRNA was moderately expressed in aortas. In 15 week old spontaneously hypertensive-stroke prone rats, there was trend toward upregulation of 6585 mRNA compared to Wistar Kyoto normotensive controls (not significant, p=0.0527).

6585 is known as acyl peptide hydrolase, a member of a group of serine peptidases from the prolyl oligopeptidase family. Prolyl oligopeptidase is involved in blood pressure control and amnesia (Polgar L., The prolyl oligopeptidase family. Cell Mol Life Sci February 2002;59(2):349-62). The enriched expression of the 6585 gene product in human vessels and the upregulation of 6585 in human failing myocardium as well as hypertensive rat aortas, indicates that 6585 plays multiple roles in the cardiovascular system. Specifically, antagonism of 6585 should reduce vascular tone and potentially reduce the progression of damage in congestive heart failure. Due to 6585 mRNA expression in human arteries, umbilical vein endothelial cells, coronary smooth muscle cells, hearts in congestive heart failure, kidneys, skeletal muscle and brain cortex, along with its functional role, modulators of 6585 activity would be useful in treating disorders associated with cardiovascular disease including but not limited to ischemia and heart failure. 6585 polypeptides of the present invention are useful in screening for modulators of 6585 activity.

Various aspects of the invention are described in further detail in the following subsections:

Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins, have a stimulatory or inhibitory effect on, for example, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, or have a stimulatory or inhibitory effect on, for example, the expression or activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate. Compounds identified using the assays described herein may be useful for treating cardiovascular diseases, e.g., atherosclerosis and/or thrombosis.

These assays are designed to identify compounds that bind to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, bind to other intracellular or extracellular proteins that interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, and interfere with the interaction of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein with other intercellular or extracellular proteins. For example, in the case of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, which is a transmembrane receptor-type protein, such techniques can identify ligands for such a receptor. A 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein ligand or substrate can, for example, be used to ameliorate cardiovascular diseases, e.g., atherosclerosis, ischemia/reperfusion, hypertension, restenosis, arterial inflammation, thrombosis and endothelial cell disorders. Such compounds may include, but are not limited to peptides, antibodies, or small organic or inorganic compounds. Such compounds may also include other cellular proteins.

Compounds identified via assays such as those described herein may be useful, for example, for ameliorating cardiovascular disease, e.g., athersclerosis and/or thrombosis. In instances whereby a cardiovascular disease condition results from an overall lower level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression and/or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein in a cell or tissue, compounds that interact with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein may include compounds which accentuate or amplify the activity of the bound 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Such compounds would bring about an effective increase in the level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein activity, thus ameliorating symptoms.

In other instances, mutations within the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene may cause aberrant types or excessive amounts of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins to be made which have a deleterious effect that leads to a cardiovascular disease. Similarly, physiological conditions may cause an excessive increase in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression leading to a cardiovascular disease. In such cases, compounds that bind to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein may be identified that inhibit the activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Assays for testing the effectiveness of compounds identified by techniques such as those described in this section are discussed herein.

In one embodiment, the invention provides assays for screening candidate or test compounds which are substrates of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or polypeptide or biologically active portion thereof. In another embodiment, the invention provides assays for screening candidate or test compounds which bind to or modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof is contacted with a test compound and the ability of the test compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is determined. Determining the ability of the test compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with angiogenesis, or the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-regulated transcription factor. The cell can be of mammalian origin, e.g., an endothelial cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of cardiovascular disease.

The ability of the test compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 binding to a substrate or to bind to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can also be determined. Determining the ability of the test compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 binding to a substrate can be accomplished, for example, by coupling the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate with a radioisotope or enzymatic label such that binding of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be determined by detecting the labeled 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate in a complex. 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 could also be coupled with a radioisotope or enzymatic label to monitor the ability of a test compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 binding to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate in a complex. Determining the ability of the test compound to bind 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be accomplished, for example, by coupling the compound with a radioisotope or enzymatic label such that binding of the compound to 1682, 6169, 6193, 7771, 14395, 29002, 33216 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be determined by detecting the labeled 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 compound in a complex. For example, compounds (e.g., 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 ligand or substrate) to interact with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 without the labeling of any of the interactants. For example, a microphysiometer can be used to detect the interaction of a compound with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 without the labeling of either the compound or the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585.

In another embodiment, an assay is a cell-based assay comprising contacting a cell expressing a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135,-10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate) with a test compound and determining the ability of the test compound to modulate (e.g., stimulate or inhibit) the activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule. Determining the ability of the test compound to modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule can be accomplished, for example, by determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to bind to or interact with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule.

Determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or a biologically active fragment thereof, to bind to or interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule can be accomplished by one of the methods described above for determining direct binding. In a preferred embodiment, determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to bind to or interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof, is contacted with a test compound and the ability of the test compound to bind to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof is determined. Preferred biologically active portions of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins to be used in assays of the present invention include fragments which participate in interactions with non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecules, e.g., fragments with high surface probability scores. Binding of the test compound to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be determined either directly or indirectly as described above. In a preferred embodiment, the assay includes contacting the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof with a known compound which binds 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 to form an assay mixture, contacting the assay mixture with a test compound, and determining the ability of the test compound to interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, wherein determining the ability of the test compound to interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein comprises determining the ability of the test compound to preferentially bind to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 or biologically active portion thereof as compared to the known compound. Compounds that modulate the interaction of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 with a known target protein may be useful in regulating the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, especially a mutant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

In another embodiment, the assay is a cell-free assay in which a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof is contacted with a test compound and the ability of the test compound to modulate (e.g., stimulate or inhibit) the activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof is determined. Determining the ability of the test compound to modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be accomplished, for example, by determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to bind to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule by one of the methods described above for determining direct binding. Determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to bind to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be accomplished by determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to further modulate the activity of a downstream effector of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule. For example, the activity of the effector molecule on an appropriate target can be determined or the binding of the effector to an appropriate target can be determined as previously described.

In yet another embodiment, the cell-free assay involves contacting a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or biologically active portion thereof with a known compound which binds the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to form an assay mixture, contacting the assay mixture with a test compound, and determining the ability of the test compound to interact with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, wherein determining the ability of the test compound to interact with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein comprises determining the ability of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to preferentially bind to or modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule.

In more than one embodiment of the above assay methods of the present invention, it may be desirable to immobilize either 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 or its target molecule to facilitate separation of complexed from uncomplexed forms of one or both of the proteins, as well as to accommodate automation of the assay. Binding of a test compound to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, or interaction of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 binding or activity determined using standard techniques.

Other techniques for immobilizing proteins on matrices can also be used in the screening assays of the invention. For example, either a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 target molecule can be immobilized utilizing conjugation of biotin and streptavidin. Biotinylated 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or target molecules but which do not interfere with binding of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to its target molecule can be derivatized to the wells of the plate, and unbound target or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein trapped in the wells by antibody conjugation. Methods for detecting such complexes, in addition to those described above for the GST-immobilized complexes, include immunodetection of complexes using antibodies reactive with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or target molecule, as well as enzyme-linked assays which rely on detecting an enzymatic activity associated with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or target molecule.

In another embodiment, modulators of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression are identified in a method wherein a cell is contacted with a candidate compound and the expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein in the cell is determined. The level of expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein in the presence of the candidate compound is compared to the level of expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein in the absence of the candidate compound. The candidate compound can then be identified as a modulator of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression based on this comparison. For example, when expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein is greater (statistically significantly greater) in the presence of the candidate compound than in its absence, the candidate compound is identified as a stimulator of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein expression. Alternatively, when expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein is less (statistically significantly less) in the presence of the candidate compound than in its absence, the candidate compound is identified as an inhibitor of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein expression. The level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein expression in the cells can be determined by methods described herein for detecting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or protein.

In yet another aspect of the invention, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 (“1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-binding proteins” or “1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-bp”) and are involved in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. Such 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-binding proteins are also likely to be involved in the propagation of signals by the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 targets as, for example, downstream elements of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-mediated signaling pathway. Alternatively, such 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-binding proteins are likely to be 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 inhibitors.

The two-hybrid system is based on the modular nature of most transcription factors, which consist of separable DNA-binding and activation domains. Briefly, the assay utilizes two different DNA constructs. In one construct, the gene that codes for a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

In another aspect, the invention pertains to a combination of two or more of the assays described herein. For example, a modulating agent can be identified using a cell-based or a cell free assay, and the ability of the agent to modulate the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be confirmed in vivo, e.g., in an animal such as an animal model for cardiovascular disease, e.g., atherosclerosis and/or thrombosis, as described herein.

This invention further pertains to novel agents identified by the above-described screening assays. Accordingly, it is within the scope of this invention to further use an agent identified as described herein in an appropriate animal model. For example, an agent identified as described herein (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulating agent, an antisense 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecule, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-specific antibody, or a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to treat cardiovascular disease symptoms. Cell-based and animal model-based assays for the identification of compounds exhibiting such an ability to ameliorate cardiovascular disease systems are described herein.

In one aspect, cell-based systems, as described herein, may be used to identify compounds which may act to treat at least one cardiovascular disease symptom. For example, such cell systems may be exposed to a compound, suspected of exhibiting an ability to treat cardiovascular disease symptoms, at a sufficient concentration and for a time sufficient to elicit such an amelioration of cardiovascular disease symptoms in the exposed cells. After exposure, the cells are examined to determine whether one or more of the cardiovascular disease cellular phenotypes has been altered to resemble a more normal or more wild type, non-cardiovascular disease phenotype. Cellular phenotypes that are associated with cardiovascular disease states include aberrant proliferation and migration, angiogenesis, deposition of extracellular matrix components, accumulation of intracellular lipids, and expression of growth factors, cytokines, and other inflammatory mediators.

In addition, animal-based cardiovascular disease systems, such as those described herein, may be used to identify compounds capable of ameliorating cardiovascular disease symptoms. Such animal models may be used as test substrates for the identification of drugs, pharmaceuticals, therapies, and interventions which may be effective in treating cardiovascular disease. For example, animal models may be exposed to a compound, suspected of exhibiting an ability to ameliorate cardiovascular disease symptoms, at a sufficient concentration and for a time sufficient to elicit such an amelioration of cardiovascular disease symptoms in the exposed animals. The response of the animals to the exposure may be monitored by assessing the reversal of disorders associated with cardiovascular disease, for example, by counting the number of atherosclerotic plaques and/or measuring their size before and after treatment.

With regard to intervention, any treatments which reverse any aspect of cardiovascular disease symptoms should be considered as candidates for human cardiovascular disease therapeutic intervention. Dosages of test agents may be determined by deriving dose-response curves.

Additionally, gene expression patterns may be utilized to assess the ability of a compound to ameliorate cardiovascular disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, atherosclerosis, ischemia/reperfusion, hypertension, restenosis, and arterial inflammation, including any of the control or experimental conditions described herein, for example, atherogenic cytokine stimulation of macrophages. Gene expression profiles may be generated, for example, by utilizing a differential display procedure, Northern analysis and/or RT-PCR. In one embodiment, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences may be used as probes and/or PCR primers for the generation and corroboration of such gene expression profiles.

Gene expression profiles may be characterized for known states, either cardiovascular disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cardiovascular disease model system to more closely resemble the control system. Administration of a compound may, alternatively, cause the gene expression profile of a control system to begin to mimic a cardiovascular disease state. Such a compound may, for example, be used in further characterizing the compound of interest, or may be used in the generation of additional animal models.

Cell- and Animal-Based Model Systems

Described herein are cell- and animal-based systems which act as models for cardiovascular disease. These systems may be used in a variety of applications. For example, the cell- and animal-based model systems may be used to further characterize differentially expressed genes associated with cardiovascular disease, e.g., 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. In addition, animal- and cell-based assays may be used as part of screening strategies designed to identify compounds which are capable of ameliorating cardiovascular disease symptoms, as described, below. Thus, the animal- and cell-based models may be used to identify drugs, pharmaceuticals, therapies and interventions which may be effective in treating cardiovascular disease. Furthermore, such animal models may be used to determine the LD50 and the ED50 in animal subjects, and such data can be used to determine the in vivo efficacy of potential cardiovascular disease treatments.

Animal-Based Systems

Animal-based model systems of cardiovascular disease may include, but are not limited to, non-recombinant and engineered transgenic animals.

Non-recombinant animal models for cardiovascular disease may include, for example, genetic models. Such genetic cardiovascular disease models may include, for example, ApoB or ApoR deficient pigs (Rapacz, et al., 1986, Science 234:1573-1577) and Watanabe heritable hyperlipidemic (WHHL) rabbits (Kita et al., 1987, Proc. Natl. Acad. Sci USA 84: 5928-5931). Transgenic mouse models in cardiovascular disease and angiogenesis are reviewed in Carmeliet, P. and Collen, D. (2000) J. Pathol. 190:387-405.

Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty. Animal models of cardiovascular disease also include rat myocardial infarction models (described in, for example, Schwarz, ER et al. (2000) J. Am. Coll. Cardiol. 35:1323-1330) and models of chromic cardiac ischemia in rabbits (described in, for example, Operschall, C et al. (2000) J. Appl. Physiol. 88:1438-1445).

Additionally, animal models exhibiting cardiovascular disease symptoms may be engineered by using, for example, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences described above, in conjunction with techniques for producing transgenic animals that are well known to those of skill in the art. For example, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences may be introduced into, and overexpressed in, the genome of the animal of interest, or, if endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences are present, they may either be overexpressed or, alternatively, be disrupted in order to underexpress or inactivate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression, such as described for the disruption of ApoE in mice (Plump et al., 1992, Cell 71: 343-353).

The host cells of the invention can also be used to produce non-human transgenic animals. For example, in one embodiment, a host cell of the invention is a fertilized oocyte or an embryonic stem cell into which 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-coding sequences have been introduced. Such host cells can then be used to create non-human transgenic animals in which exogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences have been introduced into their genome or homologous recombinant animals in which endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences have been altered. Such animals are useful for studying the function and/or activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 and for identifying and/or evaluating modulators of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding nucleic acid into the male pronuclei of a fertilized oocyte, e.g., by microinjection, retroviral infection, and allowing the oocyte to develop in a pseudopregnant female foster animal. The 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 cDNA sequence can be introduced as a transgene into the genome of a non-human animal. Alternatively, a nonhuman homologue of a human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, such as a mouse or rat 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, can be used as a transgene. Alternatively, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene homologue, such as another 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 family member, can be isolated based on hybridization to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 cDNA sequences and used as a transgene. Intronic sequences and polyadenylation signals can also be included in the transgene to increase the efficiency of expression of the transgene. A tissue-specific regulatory sequence(s) can be operably linked to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 transgene to direct expression of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 transgene in its genome and/or expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA in tissues or cells of the animals. A transgenic founder animal can then be used to breed additional animals carrying the transgene. Moreover, transgenic animals carrying a transgene encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can further be bred to other transgenic animals carrying other transgenes.

To create a homologous recombinant animal, a vector is prepared which contains at least a portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene into which a deletion, addition or substitution has been introduced to thereby alter, e.g., functionally disrupt, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene. The 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene can be a human gene but more preferably, is a non-human homologue of a human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene. For example, a rat 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene can be used to construct a homologous recombination nucleic acid molecule, e.g., a vector, suitable for altering an endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene in the mouse genome. In a preferred embodiment, the homologous recombination nucleic acid molecule is designed such that, upon homologous recombination, the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene is functionally disrupted (i.e., no longer encodes a functional protein; also referred to as a “knock out” vector). Alternatively, the homologous recombination nucleic acid molecule can be designed such that, upon homologous recombination, the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene is mutated or otherwise altered but still encodes functional protein (e.g., the upstream regulatory region can be altered to thereby alter the expression of the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein). In the homologous recombination nucleic acid molecule, the altered portion of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene is flanked at its 5′ and 3′ ends by additional nucleic acid sequence of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene to allow for homologous recombination to occur between the exogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene carried by the homologous recombination nucleic acid molecule and an endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene in a-cell, e.g., an embryonic stem cell. The additional flanking 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene has homologously recombined with the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijistra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 transgenic animals that express 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 peptide (detected immunocytochemically, using antibodies directed against 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 epitopes) at easily detectable levels should then be further evaluated to identify those animals which display characteristic cardiovascular disease symptoms. Such cardiovascular disease symptoms may include, for example, increased prevalence and size of fatty streaks and/or cardiovascular disease plaques.

Additionally, specific cell types (e.g., endothelial cells) within the transgenic animals may be analyzed and assayed for cellular phenotypes characteristic of cardiovascular disease. In the case of endothelial cells, such phenotypes include, but are not limited to cell proliferation, migration, angiogenesis, production of proinflammatory growth factors and cytokines, and adhesion to inflammatory cells. In the case of monocytes, such phenotypes may include but are not limited to increases in rates of LDL uptake, adhesion to endothelial cells, transmigration, foam cell formation, fatty streak formation, and production of foam cell specific products. Cellular phenotypes may include a particular cell type's pattern of expression of genes associated with cardiovascular disease as compared to known expression profiles of the particular cell type in animals exhibiting cardiovascular disease symptoms.

Cell-Based Systems

Cells that contain and express 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences which encode a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, and, further, exhibit cellular phenotypes associated with cardiovascular disease, may be used to identify compounds that exhibit anti-cardiovascular disease activity. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651). Further, such cells may include recombinant, transgenic cell lines. For example, the cardiovascular disease animal models of the invention, discussed above, may be used to generate cell lines, containing one or more cell types involved in cardiovascular disease, that can be used as cell culture models for this disorder. While primary cultures derived from the cardiovascular disease transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in cardiovascular disease may be transfected with sequences capable of increasing or decreasing the amount of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression within the cell. For example, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences may be introduced into, and overexpressed in, the genome of the cell of interest, or, if endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences are present, they may be either overexpressed or, alternatively disrupted in order to underexpress or inactivate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression.

In order to overexpress a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, the coding portion of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequence, such a sequence may be isolated and engineered such that when reintroduced into the genome of the cell type of interest, the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 alleles will be inactivated. Preferably, the engineered 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence is introduced via gene targeting such that the endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence is disrupted upon integration of the engineered 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence into the cell's genome. Transfection of host cells with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 5959.0, 15992, 2094, 2252, 3474, 9792, 15400, 1452 or 6585 genes is discussed, above.

Cells treated with compounds or transfected with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 genes can be examined for phenotypes associated with cardiovascular disease. In the case of monocytes, such phenotypes include but are not limited to increases in rates of LDL uptake, adhesion to endothelial cells, transmigration, foam cell formation, fatty streak formation, and production by foam cells of growth factors such as bFGF, IGF-I, VEGF, IL-1, M-CSF, TGFβ, TGFα, TNFα, HB-EGF, PDGF, IFN-γ, and GM-CSF. Transmigration rates, for example, may be measured using the in vitro system of Navab et al. (1988) J. Clin. Invest. 82:1853-1863, by quantifying the number of monocytes that migrate across the endothelial monolayer and into the collagen layer of the subendothelial space.

Similarly, endothelial cells can be treated with test compounds or transfected with genetically engineered 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 genes. The endothelial cells can then be examined for phenotypes associated with cardiovascular disease, including, but not limited to changes in cellular morphology, cell proliferation, cell migration, and mononuclear cell adhesion; or for the effects on production of other proteins involved in cardiovascular disease such as adhesion molecules (e.g., ICAM, VCAM, E-selectin), growth factors and cytokines (e.g., PDGF, IL-1β, TNFα, MCF), and proteins involved in angiogenesis (e.g., FLK, FLT).

Transfection of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid may be accomplished by using standard techniques (described in, for example, Ausubel (1989) supra). Transfected cells should be evaluated for the presence of the recombinant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences, for expression and accumulation of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA, and for the presence of recombinant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein production. In instances wherein a decrease in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression is desired, standard techniques may be used to demonstrate whether a decrease in endogenous 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression and/or in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein production is achieved.

Cellular models for the study of cardiovascular disease and angiogenesis include models of endothelial cell differentiation on Matrigel (Baatout, S. et al. (1996) Rom. J. Intern. Med. 34:263-269; Benelli, R et al. (1999) Int. J. Biol. Markers 14:243-246), embryonic stem cell models of vascular morphogenesis (Doetschman, T. et al. (1993) Hypertension 22:618-629), the culture of microvessel fragments in physiological gels (Hoying, J B et al. (1996) In Vitro Cell Dev. Biol. Anim. 32: 409-419; U.S. Pat. No. 5,976,782), and the treatment of endothelial cells and smooth muscle cells with atherogenic and angiogenic factors including growth factors and cytokines (e.g., IL-1β, PDGF, TNFα, VEGF), homocysteine, and LDL. In vitro angiogenesis models are described in, for example, Black, A F et al. (1999) Cell Biol. Toxicol. 15:81-90.

Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein and/or nucleic acid expression as well as 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, in the context of a biological sample (e.g., blood, serum, cells, e.g., endothelial cells, or tissue, e.g., vascular tissue) to thereby determine whether an individual is afflicted with a cardiovascular disease. The invention also provides for prognostic (or predictive) assays for determining whether an individual is at risk of developing a cardiovascular disorder. For example, mutations in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene can be assayed for in a biological sample. Such assays can be used for prognostic or predictive purpose to thereby phophylactically treat an individual prior to the onset of a cardiovascular disorder, e.g., atherosclerosis.

Another aspect of the invention pertains to monitoring the influence of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulators (e.g., anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 ribozymes) on the expression or activity of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 in clinical trials.

These and other agents are described in further detail in the following sections.

Diagnostic Assays For Cardiovascular Disease

To determine whether a subject is afflicted with a cardiovascular disease, a biological sample may be obtained from a subject and the biological sample may be contacted with a compound or an agent capable of detecting a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or nucleic acid (e.g., mRNA or genomic DNA) that encodes a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, in the biological sample. A preferred agent for detecting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or genomic DNA is a labeled nucleic acid probe capable of hybridizing to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or genomic DNA. The nucleic acid probe can be, for example, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or a portion thereof, such as an oligonucleotide of at least 15, 20, 25, 30, 25, 40, 45, 50, 100, 250 or 500 nucleotides in length and sufficient to specifically hybridize under stringent conditions to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA or genomic DNA. Other suitable probes for use in the diagnostic assays of the invention are described herein.

A preferred agent for detecting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein in a sample is an antibody capable of binding to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA, protein, or genomic DNA in a biological sample in vitro as well as in vivo. For example, in vitro techniques for detection of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA include Northern hybridizations and in situ hybridizations. In vitro techniques for detection of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein include enzyme linked immunosorbent assays (ELISAs), Western blots, immunoprecipitations and immunofluorescence. In vitro techniques for detection of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 genomic DNA include Southern hybridizations. Furthermore, in vivo techniques for detection of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein include introducing into a subject a labeled anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA, or genomic DNA, such that the presence of 1682, 6169, 6193, 7771, 14395, 29002, 33216, 43726, 69292, 21656, 32427, 2402, 7747, 1720, 9151, 60491, 1371, 7077, 33207, 1419, 18, 036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA or genomic DNA is detected in the biological sample, and comparing the presence of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA or genomic DNA in the control sample with the presence of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA or genomic DNA in the test sample.

Prognostic Assays for Cardiovascular Disease

The present invention further pertains to methods for identifying subjects having or at risk of developing a cardiovascular disease associated with aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity.

As used herein, the term “aberrant” includes a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity which deviates from the wild type 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity. Aberrant expression or activity includes increased or decreased expression or activity, as well as expression or activity which does not follow the wild type developmental pattern of expression or the subcellular pattern of expression. For example, aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity is intended to include the cases in which a mutation in the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene causes the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene to be under-expressed or over-expressed and situations in which such mutations result in a non-functional 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or a protein which does not function in a wild-type fashion, e.g., a protein which does not interact with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate, or one which interacts with a non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate.

The assays described herein, such as the preceding diagnostic assays or the following assays, can be used to identify a subject having or at risk of developing a cardiovascular disease, e.g., including but not limited to, atherosclerosis, ischemia/reperfusion injury, hypertension, restenosis, arterial inflammation, and endothelial cell disorders. A biological sample may be obtained from a subject and tested for the presence or absence of a genetic alteration. For example, such genetic alterations can be detected by ascertaining the existence of at least one of 1) a deletion of one or more nucleotides from a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 2) an addition of one or more nucleotides to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 3) a substitution of one or more nucleotides of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 4) a chromosomal rearrangement of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 5) an alteration in the level of a messenger RNA transcript of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 6) aberrant modification of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, such as of the methylation pattern of the genomic DNA, 7) the presence of a non-wild type splicing pattern of a messenger RNA transcript of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, 8) a non-wild type level of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-protein, 9) allelic loss of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, and 10) inappropriate post-translational modification of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-protein.

As described herein, there are a large number of assays known in the art which can be used for detecting genetic alterations in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene. For example, a genetic alteration in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene under conditions such that hybridization and amplification of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene in a biological sample and detect mutations by comparing the sequence of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence, e.g., a wild-type 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator (e.g., an agonist, antagonist, peptidomimetic, protein, peptide, nucleic acid, or small molecule) to effectively treat a cardiovascular disease, e.g., atherosclerosis.

Monitoring of Effects During Clinical Trials

The present invention further provides methods for determining the effectiveness of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator identified herein) in treating a cardiovascular disease, e.g., atherosclerosis and/or thrombosis, in a subject. For example, the effectiveness of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator in increasing 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression, protein levels, or in upregulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, can be monitored in clinical trials of subjects exhibiting decreased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression, protein levels, or downregulated 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. Alternatively, the effectiveness of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator in decreasing 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression, protein levels, or in downregulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, can be monitored in clinical trials of subjects exhibiting increased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression, protein levels, or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. In such clinical trials, the expression or activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, and preferably, other genes that have been implicated in, for example, atherosclerosis and/or thrombosis can be used as a “read out” or marker of the phenotype of a particular cell, e.g., a vascular endothelial cell.

For example, and not by way of limitation, genes, including 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585, that are modulated in cells by treatment with an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity (e.g., identified in a screening assay as described herein) can be identified. Thus, to study the effect of agents which modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity on subjects suffering from a cardiovascular disease in, for example, a clinical trial, cells can be isolated and RNA prepared and analyzed for the levels of expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 and other genes implicated in the cardiovascular disease. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 or other genes. In this way, the gene expression pattern can serve as a marker, indicative of the physiological response of the cells to the agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. This response state may be determined before, and at various points during treatment of the individual with the agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity.

In a preferred embodiment, the present invention provides a method for monitoring the effectiveness of treatment of a subject with an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA, or genomic DNA in the pre-administration sample; (iii) obtaining one or more post-administration samples from the subject; (iv) detecting the level of expression or activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA, or genomic DNA in the post-administration samples; (v) comparing the level of expression or activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA, or genomic DNA in the pre-administration sample with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, mRNA, or genomic DNA in the post administration sample or samples; and (vi) altering the administration of the agent to the subject accordingly. For example, increased administration of the agent may be desirable to increase the expression or activity of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 to higher levels than detected, i.e., to increase the effectiveness of the agent. Alternatively, decreased administration of the agent may be desirable to decrease expression or activity of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 to lower levels than detected, i.e. to decrease the effectiveness of the agent. According to such an embodiment, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity may be used as an indicator of the effectiveness of an agent, even in the absence of an observable phenotypic response.

Methods of Treatment of Subjects Suffering From Cardiovascular Disease:

The present invention provides for both prophylactic and therapeutic methods of treating a subject, e.g., a human, at risk of (or susceptible to) a cardiovascular disease such as atherosclerosis, ischemia/reperfusion injury, hypertension, restenosis, arterial inflammation, thrombosis, and endothelial cell disorders. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecules of the present invention or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Prophylactic Methods

In one aspect, the invention provides a method for preventing in a subject, a cardiovascular disease by administering to the subject an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, e.g., modulation of calcium influx, cellular migration, or formation of atherosclerotic lesions. Subjects at risk for a cardiovascular disease, e.g., atherosclerosis and/or thrombosis, can be identified by, for example, any or a combination of the diagnostic or prognostic assays described herein. Administration of a prophylactic agent can occur prior to the manifestation of symptoms characteristic of aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity, such that a cardiovascular disease is prevented or, alternatively, delayed in its progression. Depending on the type of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 aberrancy, for example, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 agonist or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antagonist agent can be used for treating the subject. The appropriate agent can be determined based on screening assays described herein.

Therapeutic Methods

Described herein are methods and compositions whereby cardiovascular disease symptoms may be ameliorated. Certain cardiovascular diseases are brought about, at least in part, by an excessive level of a gene product, or by the presence of a gene product exhibiting an abnormal or excessive activity. As such, the reduction in the level and/or activity of such gene products would bring about the amelioration of cardiovascular disease symptoms. Techniques for the reduction of gene expression levels or the activity of a protein are discussed below.

Alternatively, certain other cardiovascular diseases are brought about, at least in part, by the absence or reduction of the level of gene expression, or a reduction in the level of a protein's activity. As such, an increase in the level of gene expression and/or the activity of such proteins would bring about the amelioration of cardiovascular disease symptoms.

In some cases, the up-regulation of a gene in a disease state reflects a protective role for that gene product in responding to the disease condition. Enhancement of such a gene's expression, or the activity of the gene product, will reinforce the protective effect it exerts. Some cardiovascular disease states may result from an abnormally low level of activity of such a protective gene. In these cases also, an increase in the level of gene expression and/or the activity of such gene products would bring about the amelioration of cardiovascular disease symptoms. Techniques for increasing target gene expression levels or target gene product activity levels are discussed herein.

Accordingly, another aspect of the invention pertains to methods of modulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity for therapeutic purposes. Accordingly, in an exemplary embodiment, the modulatory method of the invention involves contacting a cell with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 or agent that modulates one or more of the activities of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein activity associated with the cell (e.g., an endothelial cell or an ovarian cell). An agent that modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein activity can be an agent as described herein, such as a nucleic acid or a protein, a naturally-occurring target molecule of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 ligand or substrate), a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 agonist or antagonist, a peptidomimetic of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 agonist or antagonist, or other small molecule. In one embodiment, the agent stimulates one or more 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activities. Examples of such stimulatory agents include active 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein and a nucleic acid molecule encoding 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 that has been introduced into the cell. In another embodiment, the agent inhibits one or more 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252,<9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activities. Examples of such inhibitory agents include antisense 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules, anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies, and 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or nucleic acid molecule. In one embodiment, the method involves administering an agent (e.g., an agent identified by a screening assay described herein), or combination of agents that modulates (e.g., upregulates or downregulates) 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity. In another embodiment, the method involves administering a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or nucleic acid molecule as therapy to compensate for reduced, aberrant, or unwanted 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity.

Stimulation of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is desirable in situations in which 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 is abnormally downregulated and/or in which increased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is likely to have a beneficial effect. Likewise, inhibition of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is desirable in situations in which 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 is abnormally upregulated and/or in which decreased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is likely to have a beneficial effect.

Methods for Inhibiting Target Gene Expression, Synthesis, or Activity

As discussed above, genes involved in cardiovascular disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate cardiovascular disease symptoms. Such molecules may include, but are not limited to, small organic molecules, peptides, antibodies, and the like.

For example, compounds can be administered that compete with endogenous ligand for the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. The resulting reduction in the amount of ligand-bound 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein will modulate endothelial cell physiology. Compounds that can be particularly useful for this purpose include, for example, soluble proteins or peptides, such as peptides comprising one or more of the extracellular domains, or portions and/or analogs thereof, of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 receptor site, but do not activate the protein, (e.g., receptor-ligand antagonists) can be effective in inhibiting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein activity.

Further, antisense and ribozyme molecules which inhibit expression of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene may also be used in accordance with the invention to inhibit aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene activity. Still further, triple helix molecules may be utilized in inhibiting aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene activity.

The antisense nucleic acid molecules used in the methods of the invention are typically administered to a subject or generated in situ such that they hybridize with or bind to cellular mRNA and/or genomic DNA encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA transcripts to thereby inhibit translation of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA. A ribozyme having specificity for a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding nucleic acid can be designed based upon the nucleotide sequence of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 cDNA disclosed herein (i.e., SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125.) For example, a derivative of a Tetrahymena L-19 IVS RNA can be constructed in which the nucleotide sequence of the active site is complementary to the nucleotide sequence to be cleaved in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding mRNA (see, for example, Cech et al. U.S. Pat. No. 4,987,071; and Cech et al. U.S. Pat. No. 5,116,742). Alternatively, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA can be used to select a catalytic RNA having a specific ribonuclease activity from a pool of RNA molecules (see, for example, Bartel, D. and Szostak, J. W. (1993) Science 261:1411-1418).

1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression can also be inhibited by targeting nucleotide sequences complementary to the regulatory region of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 (e.g., the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 promoter and/or enhancers) to form triple helical structures that prevent transcription of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein and interfere with its activity may also be used to modulate or inhibit 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein function. Such antibodies may be generated using standard techniques described herein, against the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory

fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using

methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Antibodies that are specific for one or more extracellular domains of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, for example, and that interfere with its activity, are particularly useful in treating cardiovascular disease. Such antibodies are especially efficient because they can access the target domains directly from the bloodstream. Any of the administration techniques described below which are appropriate for peptide administration may be utilized to effectively administer inhibitory target gene antibodies to their site of action.

Methods for Restoring or Enhancing Target Gene Activity

Genes that cause cardiovascular disease may be underexpressed within cardiovascular disease situations. Alternatively, the activity of the protein products of such genes may be decreased, leading to the development of cardiovascular disease symptoms. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to cardiovascular disease conditions.

Described in this section are methods whereby the level 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity may be increased to levels wherein cardiovascular disease symptoms are ameliorated. The level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity may be increased, for example, by either increasing the level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene expression or by increasing the level of active 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein which is present.

For example, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, at a level sufficient to ameliorate cardiovascular disease symptoms may be administered to a patient exhibiting such symptoms. Any of the techniques discussed below may be used for such administration. One of skill in the art will readily know how to determine the concentration of effective, non-toxic doses of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, utilizing techniques such as those described below.

Additionally, RNA sequences encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein may be directly administered to a patient exhibiting cardiovascular disease symptoms, at a concentration sufficient to produce a level of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein such that cardiovascular disease symptoms are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene, or a portion thereof, that directs the production of a normal 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene sequences into human cells.

Cells, preferably, autologous cells, containing 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expressing gene sequences may then be introduced or reintroduced into the subject at positions which allow for the amelioration of cardiovascular disease symptoms. Such cell replacement techniques may be preferred, for example, when the gene product is a secreted, extracellular gene product.

Pharmaceutical Compositions

Another aspect of the invention pertains to methods for treating a subject suffering from a cardiovascular disease, e.g., atherosclerosis. These methods involve administering to a subject an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity (e.g., an agent identified by a screening assay described herein), or a combination of such agents. In another embodiment, the method involves administering to a subject a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or nucleic acid molecule as therapy to compensate for reduced, aberrant, or unwanted 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 expression or activity.

Stimulation of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is desirable in situations in which 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 is abnormally downregulated and/or in which increased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is likely to have a beneficial effect. Likewise, inhibition of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is desirable in situations in which 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 is abnormally upregulated and/or in which decreased 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity is likely to have a beneficial effect, e.g., inhibition of atherosclerotic lesion formation.

The agents which modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity (e.g., a fragment of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or an anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity can also be prepared in the form of suppositories (e.g., with conventional suppository bases such as cocoa butter and other glycerides) or retention enemas for rectal delivery.

In one embodiment, the agents that modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0. 1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is

Furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouraci1 decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity, as well as tailoring the dosage and/or therapeutic regimen of treatment with an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzylmopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecule or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cardiovascular disease, e.g., atherosclerosis, with an agent which modulates 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity.

Recombinant Expression Vectors and Host Cells Used in the Methods of the Invention

The methods of the invention (e.g., the screening assays described herein) include the use of vectors, preferably expression vectors, containing a nucleic acid encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins, mutant forms of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins, fusion proteins, and the like).

The recombinant expression vectors to be used in the methods of the invention can be designed for expression of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins in prokaryotic or eukaryotic cells. For example, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. Coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity assays, (e.g., direct assays or competitive assays described in detail below), or to generate antibodies specific for 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins. In a preferred embodiment, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecule of the invention is introduced, e.g., a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecule within a recombinant expression vector or a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Accordingly, the invention further provides methods for producing a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein using the host cells of the invention. In one embodiment, the method comprises culturing the host cell of the invention (into which a recombinant expression vector encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein has been introduced) in a suitable medium such that a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein is produced. In another embodiment, the method further comprises isolating a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein from the medium or the host cell.

Isolated Nucleic Acid Molecules Used in the Methods of the Invention

The methods of the invention include the use of isolated nucleic acid molecules that encode 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins or biologically active portions thereof, as well as nucleic acid fragments sufficient for use as hybridization probes to identify 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding nucleic acid molecules (e.g., 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA) and fragments for use as PCR primers for the amplification or mutation of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or a portion thereof, can be isolated using standard molecular biology techniques and the sequence information provided herein. Using all or portion of the nucleic acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125 as a hybridization probe, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, a complement of the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or a portion of any of these nucleotide sequences. A nucleic acid molecule which is complementary to the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125 is one which is sufficiently complementary to the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or a portion of any of this nucleotide sequence.

Moreover, the nucleic acid molecules used in the methods of the invention can comprise only a portion of the nucleic acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, for example, a fragment which can be used as a probe or primer or a fragment encoding a portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, e.g., a biologically active portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or of a naturally occurring allelic variant or mutant of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in IX SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0. 15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na+] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, such as by measuring a level of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding nucleic acid in a sample of cells from a subject e.g., detecting 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA levels or determining whether a genomic 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene has been mutated or deleted.

The methods of the invention further encompass the use of nucleic acid molecules that differ from the nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, due to degeneracy of the genetic code and thus encode the same 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126.

The methods of the invention further include the use of allelic variants of human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 e.g., functional and non-functional allelic variants. Functional allelic variants are naturally occurring amino acid sequence variants of the human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein that maintain a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126 or substitution, deletion or insertion of non-critical residues in non-critical regions of the protein.

Non-functional allelic variants are naturally occurring amino acid sequence variants of the human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein that do not have a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126, or a substitution, insertion or deletion in critical residues or critical regions of the protein.

The methods of the present invention may further use non-human orthologues of the human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Orthologues of the human 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein are proteins that are isolated from non-human organisms and possess the same 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity.

The methods of the present invention further include the use of nucleic acid molecules comprising the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126) without altering the biological activity, whereas an “essential” amino acid residue is required for biological activity. For example, amino acid residues that are conserved among the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins of the present invention and other members of the family are not likely to be amenable to alteration.

Mutations can be introduced into SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein is preferably replaced with another amino acid residue from the same side chain family. Alternatively, in another embodiment, mutations can be introduced randomly along all or part of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 coding sequence, such as by saturation mutagenesis, and the resultant mutants can be screened for 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123 or 125. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 coding strand, or to only a portion thereof. In one embodiment, an antisense nucleic acid molecule is antisense to a “coding region” of the coding strand of a nucleotide sequence encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. The term “noncoding region” refers to 5′ and 3′ sequences which flank the coding region that are not translated into amino acids (also referred to as 5′ and 3′ untranslated regions).

Given the coding strand sequences encoding 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 disclosed herein, antisense nucleic acids of the invention can be designed according to the rules of Watson and Crick base pairing. The antisense nucleic acid molecule can be complementary to the entire coding region of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA, but more preferably is an oligonucleotide which is antisense to only a portion of the coding or noncoding region of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA. For example, the antisense oligonucleotide can be complementary to the region surrounding the translation start site of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 139, 258, 1261, 1486, 2398, 2414, 7660, 8587, 10183, 10550, 12680, 17921, 32248, 60489 or 93804 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'eefe et al. (1996) Proc. Natl. Acad. Sci. 93:14670-675.

PNAs of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules can also be used in the analysis of single base pair mutations in a gene, (e.g., by PNA-directed PCR clamping); as ‘artificial restriction enzymes’ when used in combination with other enzymes, (e.g., SI nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Isolated 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400. 1452 or 6585 Proteins and Anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 Antibodies Used in the Methods of the Invention

The methods of the invention include the use of isolated 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins, and biologically active portions thereof, as well as polypeptide fragments suitable for use as immunogens to raise anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies. In one embodiment, native 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins can be isolated from cells or tissue sources by an appropriate purification scheme using standard protein purification techniques. In another embodiment, 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins are produced by recombinant DNA techniques. Alternative to recombinant expression, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or polypeptide can be synthesized chemically using standard peptide synthesis techniques.

As used herein, a “biologically active portion” of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein includes a fragment of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein having a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity. Biologically active portions of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein include peptides comprising amino acid sequences sufficiently identical to or derived from the amino acid sequence of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126 which include fewer amino acids than the full length 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins, and exhibit at least one activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Typically, biologically active portions comprise a domain or motif with at least one activity of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein (e.g., the N-terminal region of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein that is believed to be involved in the regulation of apoptotic activity). A biologically active portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be a polypeptide which is, for example, 25, 50, 75, 100, 125, 150, 175, 200, 250, 300 or more amino acids in length. Biologically active portions of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be used as targets for developing agents which modulate a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 activity.

In a preferred embodiment, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126. In other embodiments, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126, yet differs in amino acid sequence due to natural allelic variation or mutagenesis, as described in detail in subsection V above. Accordingly, in another embodiment, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126, having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 chimeric or fusion proteins. As used herein, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 “chimeric protein” or “fusion protein” comprises a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide operatively linked to a non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide. An “1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide” refers to a polypeptide having an amino acid sequence corresponding to a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecule, whereas a “non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide” refers to a polypeptide having an amino acid sequence corresponding to a protein which is not substantially homologous to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, e.g., a protein which is different from the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein and which is derived from the same or a different organism. Within a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion protein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide can correspond to all or a portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. In a preferred embodiment, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion protein comprises at least one biologically active portion of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. In another preferred embodiment, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion protein comprises at least two biologically active portions of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Within the fusion protein, the term “operatively linked” is intended to indicate that the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide and the non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide are fused in-frame to each other. The non-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide can be fused to the N-terminus or C-terminus of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide.

For example, in one embodiment, the fusion protein is a GST-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion protein in which the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences are fused to the C-terminus of the GST sequences. Such fusion proteins can facilitate the purification of recombinant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585.

In another embodiment, this fusion protein is a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein containing a heterologous signal sequence at its N-terminus. In certain host cells (e.g., mammalian host cells), expression and/or secretion of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be increased through use of a heterologous signal sequence.

The 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion proteins used in the methods of the invention can be incorporated into pharmaceutical compositions and administered to a subject in vivo. The 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion proteins can be used to affect the bioavailability of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate. Use of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fusion proteins may be useful therapeutically for the treatment of disorders caused by, for example, (i) aberrant modification or mutation of a gene encoding a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein; (ii) mis-regulation of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 gene; and (iii) aberrant post-translational modification of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

Moreover, the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-fusion proteins used in the methods of the invention can be used as immunogens to produce anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies in a subject, to purify 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 ligands and in screening assays to identify molecules which inhibit the interaction of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 substrate.

Preferably, a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-encoding nucleic acid can be cloned into such an expression vector such that the fusion moiety is linked in-frame to the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

The present invention also pertains to the use of variants of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins which function as either 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 agonists (mimetics) or as 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antagonists. Variants of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins can be generated by mutagenesis, e.g., discrete point mutation or truncation of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. An agonist of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins can retain substantially the same, or a subset, of the biological activities of the naturally occurring form of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. An antagonist of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can inhibit one or more of the activities of the naturally occurring form of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein by, for example, competitively modulating a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585-mediated activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

In one embodiment, variants of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein which function as either 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 agonists (mimetics) or as 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antagonists can be identified by screening combinatorial libraries of mutants, e.g., truncation mutants, of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein for 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein agonist or antagonist activity. In one embodiment, a variegated library of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 variants is generated by combinatorial mutagenesis at the nucleic acid level and is encoded by a variegated gene library. A variegated library of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 variants can be produced by, for example, enzymatically ligating a mixture of synthetic oligonucleotides into gene sequences such that a degenerate set of potential 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences is expressible as individual polypeptides, or alternatively, as a set of larger fusion proteins (e.g., for phage display) containing the set of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences therein. There are a variety of methods which can be used to produce libraries of potential 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein coding sequence can be used to generate a variegated population of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 fragments for screening and subsequent selection of variants of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. In one embodiment, a library of coding sequence fragments can be generated by treating a double stranded PCR fragment of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies. An isolated 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein, or a portion or fragment thereof, can be used as an immunogen to generate antibodies that bind 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 using standard techniques for polyclonal and monoclonal antibody preparation. A full-length 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein can be used or, alternatively, antigenic peptide fragments of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be used as immunogens. The antigenic peptide of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124 or 126, and encompasses an epitope of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 such that an antibody raised against the peptide forms a specific immune complex with the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 that are located on the surface of the protein, e.g., hydrophilic regions, as well as regions with high antigenicity.

A 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 immunogen is typically used to prepare antibodies by immunizing a suitable subject, (e.g., rabbit, goat, mouse, or other mammal) with the immunogen. An appropriate immunogenic preparation can contain, for example, recombinantly expressed 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein or a chemically synthesized 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide. The preparation can further include an adjuvant, such as Freund's complete or incomplete adjuvant, or similar immunostimulatory agent. Immunization of a suitable subject with an immunogenic 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 preparation induces a polyclonal anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 molecules. The term “monoclonal antibody” or “monoclonal antibody composition”, as used herein, refers to a population of antibody molecules that contain only one species of an antigen binding site capable of immunoreacting with a particular epitope of 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. A monoclonal antibody composition thus typically displays a single binding affinity for a particular 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein with which it immunoreacts.

Polyclonal anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies can be prepared as described above by immunizing a suitable subject with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 immunogen. The anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody titer in the immunized subject can be monitored over time by standard techniques, such as with an enzyme linked immunosorbent assay (ELISA) using immobilized 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 If desired, the antibody molecules directed against 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 can be isolated from the mammal (e.g., from the blood) and further purified by well known techniques, such as protein A chromatography to obtain the IgG fraction. At an appropriate time after immunization, e.g., when the anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 immunogen as described above, and the culture supernatants of the resulting hybridoma cells are screened to identify a hybridoma producing a monoclonal antibody that binds 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585.

Any of the many well known protocols used for fusing lymphocytes and immortalized cell lines can be applied for the purpose of generating an anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These mycloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585, e.g., using a standard ELISA assay.

Alternative to preparing monoclonal antibody-secreting hybridomas, a monoclonal anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody can be identified and isolated by screening a recombinant combinatorial immunoglobulin library (e.g., an antibody phage display library) with 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 to thereby isolate immunoglobulin library members that bind 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using-methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibody can be used to detect 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein (e.g., in a cellular lysate or cell supernatant) in order to evaluate the abundance and pattern of expression of the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 protein. Anti-1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using Taqman™ Analysis

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating a cardiovascular disorder, comprising assaying the ability of the compound to modulate 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid expression or 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide activity, thereby identifying a compound capable of treating a cardiovascular disorder.

2. A method for identifying a compound capable of modulating lipid production comprising:
a) contacting a cell which expresses 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 with a test compound; and
b) assaying the ability of the test compound to modulate the expression of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid, or the activity of a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide, thereby identifying a compound capable of modulating lipid production.

3. A method for modulating lipid production in a cell comprising contacting a cell with a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator, thereby modulating lipid production in the cell.

4. The method of claim 2, wherein the cell is a hepatic cell.

5. The method of claim 3, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is a small organic molecule, peptide, antibody or antisense nucleic acid molecule.

6. The method of claim 3, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is capable of modulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide activity.

7. The method of claim 6, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is a small organic molecule, peptide, antibody or antisense nucleic acid molecule.

8. The method of claim 6, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is capable of modulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid expression.

9. A method for treating a subject having a cardiovascular disorder characterized by aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide activity or aberrant 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 nucleic acid expression comprising administering to the subject a 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator, thereby treating said subject having a cardiovascular disorder.

10. The method of claim 9, wherein said cardiovascular disorder is selected from the group consisting of arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders

11. The method of claim 9, wherein said 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is administered in a pharmaceutically acceptable formulation.

12. The method of claim 9, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is a small organic molecule, peptide, antibody or antisense nucleic acid molecule.

13. The method of claim 9, wherein the 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 modulator is capable of modulating 1682, 6169, 6193, 7771, 14395, 29002, 33216, 43726, 69292, 21656, 32427, 2402, 7747, 1720, 9151, 60491, 1371, 7077, 33207, 1419, 18036, 16105, 38650, 14245, 58848, 1870, 25856, 32394, 3484, 345, 9252, 9135, 10532, 18610, 8165, 2448, 2445, 64624, 84237, 8912, 2868, 283, 2554, 9464, 17799, 26686, 43848, 32135, 12208, 2914, 51130, 19489, 21833, 2917, 59590, 15992, 2094, 2252, 3474, 9792, 15400, 1452 or 6585 polypeptide activity.
